(19) United States
(12) Patent Application Publication
(10) Pub. No.: US 2013/0164307 A1
(43) Pub. Date: Jun. 27, 2013

(54) USE OF CAMELID-DERIVED VARIABLE HEAVY CHAIN VARIABLE REGIONS (VHH) TARGETING HUMAN CD18 AND ICAM-1 AS A MICROBICIDE TO PREVENT HIV-1 TRANSMISSION

(71) Applicant: Richard B. Markham, Columbia, MD (US)

(72) Inventor: Richard B. Markham, Columbia, MD (US)

(73) Assignee: THE JOHNS HOPKINS UNIVERSITY, BALTIMORE, MD (US)

(21) Appl. No.: 13/704,976

(22) PCT Filed: Jun. 17, 2011
(86) PCT No.: PCT/US11/40973

§ 371 Date: Dec. 17, 2012

Related U.S. Application Data
(60) Provisional application No. 61/355,794, filed on Jun. 17, 2010.

(51) Int. Cl. C07K 16/28; G01N 33/68; C07K 16/08

(52) U.S. Cl. CPC C07K 16/2821; C07K 16/087; G01N 33/6854
USPC 424/159.1; 530/389.1; 530/387.3; 424/172.1; 530/389.4; 435/252.3; 435/252.33; 506/9; 424/93.2

(57) ABSTRACT

The invention provides methods, compositions, and kits featuring a camelid-derived antibody for use in preventing or inhibiting a viral infection.

### CROSS-REFERENCE TO RELATED APPLICATION

This application claims the benefit of U.S. Provisional Application No. 61/355,794, filed Jun. 17, 2010, the contents of which is incorporated herein by reference in its entirety.

### STATEMENT OF RIGHTS TO INVENTIONS MADE UNDER FEDERALLY SPONSORED RESEARCH

This work was supported by grant nos. AI-55424, AI-60615, and AI-79794 from the National Institutes of Health. The Government has certain rights in this invention.

### BACKGROUND OF THE INVENTION

Recent failures of candidate microbicides that have entered Phase III clinical trials dictate the need for new strategies for microbicide development. The first generation microbicides were broadly reactive, but non-specific in their activity. While the detergent activity of nonoxynol-9 provides a basis for understanding the enhanced transmission observed with this treatment, the enhanced transmission observed with use of cellulose sulfate, the therapeutic failure of which was foreshadowed by macaque studies employing seminal plasma, was unexpected.

One approach to addressing these failures would be the development of microbicides with more specific and well-defined mechanisms of action that might be less prone to unanticipated adverse effects on the integrity of the natural barriers to transmission. The inefficiency of transmission in the unprotected setting provides testament to the effectiveness of these natural barriers, and their preservation in the face of microbicide prophylaxis is critical. Such specificity could be derived from use of small molecules that inhibit specific interactions between the virus and its receptors or that act as typical antiretroviral agents, inhibiting specific steps in the virus life cycle. Alternatively, specificity could also be derived from antibody-based approaches that target molecules involved in the transmission process. However, to the extent that such small molecule and antibody-based approaches target viral proteins, they remain vulnerable to the genetic diversity and high viral mutation frequency that have plagued the HIV-1 vaccine and therapeutics effort.

A series of host-cell derived, immutable adhesion molecules that are acquired by the virus as it buds from infected cells represent a potential Achilles Heel for HIV-1. Although not traditionally identified as receptors for the virus, multiple studies have demonstrated a role for adhesion molecules in virus infectivity and the ability of antibodies to those molecules to diminish infectivity of free virus. Targeting adhesion molecules to prevent HIV-1 transmission is also appealing because of the role these molecules play in movement of potentially infected cells across genitourinary or rectal epithelium.

The present invention is based on the inventors' discovery that antibodies directed at intercellular adhesion molecule-1 (ICAM-1) and the beta integrin lymphocyte function associated antigen-1 (LFA-1), can interrupt both cell-associated and cell-free transmission in in vitro and in vivo model systems.

### SUMMARY OF THE INVENTION

As described below, the present invention features novel agents, compositions, methods, and kits for preventing or inhibiting HIV infection.

In one aspect; the invention provides a camelid-derived antibody that specifically binds to ICAM-1, CD18, herpes simplex virus type-2 (HSV-2) glycoprotein D, or a fragment thereof. In embodiments, the antibody is isolated. In embodiments, the antibody is an antibody fragment. In embodiments, the antibody is humanized. In embodiments, the antibody inhibits viral migration in a transwell assay.

In one aspect, the invention provides a cell that produces any of the camelid-derived antibodies described herein. In embodiments, the cell is a bacterial cell that is capable of producing the antibody in situ. In related embodiments, the cell is a lactobacillus bacterial cell or an E. coli bacterial cell.

In one aspect, the invention provides a pharmaceutical composition containing any of the camelid-derived antibodies described herein. In another aspect, the invention provides a pharmaceutical composition containing a cell producing any of the camelid-derived antibodies described herein. In embodiments, the cell is a bacterial cell that is capable of producing the antibody in situ. In related embodiments, the cell is a lactobacillus bacterial cell or an E. coli bacterial cell.

In embodiments, the pharmaceutical composition is formulated for mucosal delivery.

In embodiments, the pharmaceutical composition is formulated for vaginal or rectal delivery.

In embodiments, the antibody is in an amount effective to inhibit the establishment or persistence of viral infection. In other embodiments, the antibody is in an amount effective to inhibit transepithelial transmission of a virus. In related embodiments, the virus is HIV (e.g., HIV-1) or HSV-2. In other embodiments, the virus is a cell-associated virus (e.g., HIV-1). In some embodiments, the virus is a cell-free virus (e.g., HIV-1).

In embodiments, the pharmaceutical composition further contains a camelid-derived antibody that specifically binds to CD18 (if the composition contains an anti-ICAM-1 antibody) or ICAM-1 (if the composition contains an anti-CD18 antibody).

In embodiments, the pharmaceutical composition further contains a bacterial cell that is capable of producing a camelid-derived antibody that specifically binds to CD18 (if the composition contains an anti-ICAM-1 antibody) or ICAM-1 (if the composition contains an anti-CD18 antibody) in situ.

In embodiments, the pharmaceutical composition further contains a pharmaceutically acceptable excipient, carrier, or adjuvant. In related embodiments, the pharmaceutical composition further comprises a pharmaceutically acceptable medium suitable for topical application.

In one aspect, the invention provides methods that inhibit the establishment or persistence of viral infection in a subject having or at risk of developing a viral infection. In embodiments, the methods involve contacting an epithelial cell with a camelid-derived antibody that specifically binds to ICAM-1, CD18, or HSV-2 glycoprotein. In related embodiments, the methods inhibit transepithelial transmission of the virus.

In one aspect, the invention provides methods that inhibit viral transmission in a subject having or at risk of developing a viral infection. In embodiments, the methods involve contacting an epithelial cell with a camelid-derived antibody that specifically binds to ICAM-1, CD18, or HSV-2 glycoprotein. In related embodiments, the methods inhibit transepithelial transmission of the virus.

In related embodiments, the virus is HIV (e.g., HIV-1) or HSV-2. In other embodiments, the virus is a cell-associated virus (e.g., HIV-1). In some embodiments, the virus is a cell-free virus (e.g., HIV-1).

In embodiments, the methods involve delivering the antibody to the subject using a bacterial delivery system. In related embodiments, the bacterial delivery system is a lactococcus delivery system. In other embodiments, the bacterial delivery system is an E. coli delivery system.

In embodiments, the methods further involve administering a camelid-derived antibody that specifically binds to CD18 (if originally administer an anti-ICAM-1 antibody) or ICAM-1 (if originally administer an anti-CD18 antibody).

In embodiments, the methods further involve administering a bacterial cell that is capable of producing a camelid-derived antibody that specifically binds to CD18 (if originally administer an anti-ICAM-1 antibody) or ICAM-1 (if originally administer an anti-CD18 antibody) in situ. In related embodiments, the bacterial delivery system that expresses a camelid-derived antibody that specifically binds to CD18 or ICAM-1 in situ is a lactococcus or E. coli delivery system.

In any of the above embodiments, the methods can reduce or prevents transmission of the virus across the epithelium. In related embodiments, the method reduces or prevents sexual transmission of HIV.

In one aspect, the invention provides a bacterial cell that produces a camelid-derived antibody that specifically binds to ICAM-1, CD18, or a fragment thereof in situ. In related embodiments, the invention provides for pharmaceutical compositions containing such a cell.

In one aspect, the invention provides methods for inhibiting the establishment or persistence of HIV-1 infection in a subject having or at risk of developing a viral infection. In embodiments, the methods involve contacting an epithelial cell with a camelid-derived antibody that specifically binds to CD18 and/or a camelid-derived antibody that specifically binds to ICAM-1. In related embodiments, the methods inhibit transepithelial transmission of the virus.

In one aspect, the invention provides methods for inhibiting HIV-1 transmission in a subject having or at risk of developing a viral infection. In embodiments, the methods involve contacting an epithelial cell with a camelid-derived antibody that specifically binds to CD18 and/or a camelid-derived antibody that specifically binds to ICAM-1. In related embodiments, the methods inhibit transepithelial transmission of the virus.

In one aspect, the invention provides methods for inhibiting the establishment or persistence of HSV-2 infection in a subject having or at risk of developing a viral infection. In embodiments, the methods involve contacting an epithelial cell with a camelid-derived antibody that specifically binds to HSV-2 glycoprotein D. In related embodiments, the methods inhibit transepithelial transmission of the virus.

In any of the above aspects and embodiments, the subject can be human. In embodiments, the subject is male. In embodiments, the subject is female.

In any of the above aspects and embodiments, the methods further inhibit viral infection of macrophages, T cells, and dendritic cells.

In one aspect, the invention provides methods for identifying an antibody or antibody fragment that specifically binds to ICAM-1, CD18, or HSV-2. In embodiments, the methods involve panning a phage-display library that displays at least one peptide comprising the framework regions of a camelid-derived VHH or an amino acid sequence having at least 60%, 70%, 80%, or 90% sequence identity thereto. In embodiments, the library is a camelid-derived VHH library. In embodiments, the peptides in the camelid-derived VHH library are obtained from an immunized camelid. In other embodiments, the peptides in the camelid-derived VHH library are synthetic VHH polypeptides, wherein the CDRs of the synthetic VHH polypeptides have been mutagenized.

In embodiments, the invention provides for antibodies or antibody fragments that specifically binds to ICAM-1, CD18, or HSV-2 obtained from any of the phage display libraries described herein. In related embodiments, the amino sequence of the antibody has at least 80%, 85%, 90%, 95%, or 99% homology to SEQ ID NO:2. In other embodiments, the amino sequence of the antibody has at least 80%, 85%, 90%, 95%, or 99% homology to SEQ ID NO:4.

In one aspect, the invention provides for polypeptides that specifically bind to CD18. In embodiments, the polypeptides have at least 80%, 85%, 90%, 95%, or 99% homology to SEQ ID NO:2. In other embodiments, the polypeptides have at least 80%, 85%, 90%, 95%, or 99% homology to SEQ ID NO:4.

Additional objects and advantages of the invention will be set forth in part in the description which follows, and in part will be obvious from the description, or may be learned by practice of the invention. The objects and advantages of the invention will be realized and attained by means of the elements and combinations disclosed herein, including those pointed out in the appended claims. It is to be understood that both the foregoing general description and the following detailed description are exemplary and explanatory only and are not restrictive of the invention as claimed. The accompanying drawings, which are incorporated in and constitute a part of this specification, illustrate several embodiments of the invention and, together with the description, serve to explain the principles of the invention.

### DEFINITIONS

To facilitate an understanding of the present invention, a number of terms and phrases are defined below.

As used herein, the singular forms “a”, “an”, and “the” include plural forms unless the context clearly dictates otherwise. Thus, for example, reference to “an antibody” includes reference to more than one antibody.

Unless specifically stated or obvious from context, as used herein, the term “or” is understood to be inclusive.

The term “including” is used herein to mean, and is used interchangeably with, the phrase “including but not limited to.”

As used herein, the terms “comprises,” “comprising,” “containing,” “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, antibody fragments (such as Fab, Fab′, F(ab′)2, and Fv fragments), camelid-derived VHH polypeptides and fragments thereof (e.g., truncated VHH), single chain Fv (scFv) mutants, multispecific antibodies such as bispecific antibodies generated from at least two intact antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. An antibody can be of any the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, and the like.

The term “antibody fragment” refers to a portion of an intact antibody and refers to the antigenic determining variable regions of an intact antibody. Examples of antibody fragments include, but are not limited to camelid-derived VHH polypeptide fragments (e.g., truncated VHH), Fab, Fab′, F(ab′)2, and Fv fragments, linear antibodies, single chain antibodies, and multispecific antibodies formed from antibody fragments.

A “monoclonal antibody” refers to a homogeneous antibody population involved in the highly specific recognition and binding of a single antigenic determinant, or epitope. This is in contrast to polyclonal antibodies that typically include different antibodies directed against different antigenic determinants. The term “monoclonal antibody” encompasses both intact and full-length monoclonal antibodies as well as antibody fragments (such as Fab, Fab′, F(ab′)2, Fv), camelid-derived VHH polypeptides and fragments thereof (e.g., truncated VHH), single chain (scFv) mutants, fusion proteins comprising an antibody portion, and any other modified immunoglobulin molecule comprising an antigen recognition site. Furthermore, “monoclonal antibody” refers to such antibodies made in any number of manners including but not limited to by hybridoma, phage selection, recombinant expression, and transgenic animals.

The term “camelid-derived antibody” or “camelid-derived VHH” are used interchangeably herein and refer to antibody proteins obtained from members of the camel and dromedary (Camelus bactrianus and Camelus dromaderius) family including new world members such as llama species (Lama paccos, Lama glama, and Lama vicugna), alpaca species (Vicugna pacos), guanaco species (Lama guanicoe), and vicuña species (Vicugna vicugna). In the context of the present application, “camelid-derived antibody” or “camelid-derived VHH” refer to antibodies from this family of mammals as found in nature that lack light chains, and are thus structurally distinct from the typical four chain quaternary structure having two heavy and two light chains, for antibodies from other animals. See International PCT/EP93/02214. These antibodies have three CDRs (CDR1, CDR2, and CDR3) that are interspersed between four framework regions. See Muyldermans, Rev Mol Biotech 74:277-302 (2001). The “camelid-derived antibody” or “camelid-derived VHH” is also known as a camelid nanobody, and has a molecular weight approximately one-tenth that of a human IgG molecule. These antibodies have a physical diameter of only a few nanometer, and one consequence of the small size is the ability to bind to antigenic sites that are functionally invisible to larger antibody proteins. The low molecular weight and compact size further result in the antibodies being extremely thermostable, stable to extreme pH, and to proteolytic digestion, and poorly antigenic. As with other antibodies of non-human origin, an amino acid sequence of a camelid derived antibody can be altered recombinantly to obtain a sequence that more closely resembles a human sequence, i.e., the nanobody can be “humanized”. Thus the natural low antigenicity of camelid antibodies to humans can be further reduced.

The term “humanized antibody” refers to forms of non-human (e.g. murine) antibodies that are specific immunoglobulin chains, chimeric immunoglobulins, or fragments thereof that contain minimal non-human (e.g., murine) sequences. Typically, humanized antibodies are human immunoglobulins in which residues from the complementary determining region (CDR) are replaced by residues from the CDR of a non-human species (e.g. mouse, rat, rabbit, hamster) that have the desired specificity, affinity, and capability (Jones et al., 1986, Nature, 321:522-525; Riechmann et al., 1988, Nature, 332:323-327; Verhoeyen et al., 1988, Science, 239:1534-1536). In some instances, the Fv framework region (FR) residues of a human immunoglobulin are replaced with the corresponding residues in an antibody from a non-human species that has the desired specificity, affinity, and capability. The humanized antibody can be further modified by the substitution of additional residues either in the Fv framework region and/or within the replaced non-human residues to refine and optimize antibody specificity, affinity, and/or capability. In general, the humanized antibody will comprise substantially all of at least one, and typically two or three, variable domains containing all or substantially all of the CDR regions that correspond to the non-human immunoglobulin whereas all or substantially all of the FR regions are those of a human immunoglobulin consensus sequence. The humanized antibody can also comprise at least a portion of an immunoglobulin constant region or domain (Fc), typically that of a human immunoglobulin. Examples of methods used to generate humanized antibodies are described in U.S. Pat. No. 5,225,539.

The term “human antibody” means an antibody produced by a human or an antibody having an amino acid sequence corresponding to an antibody produced by a human made using any technique known in the art. This definition of a human antibody includes intact or full-length antibodies, fragments thereof, and/or antibodies comprising at least one human heavy and/or light chain polypeptide.

The term “epitope” or “antigenic determinant” are used interchangeably herein and refer to that portion of an antigen capable of being recognized and specifically bound by a particular antibody. When the antigen is a polypeptide, epitopes can be formed both from contiguous amino acids and noncontiguous amino acids juxtaposed by tertiary folding of a protein. Epitopes formed from contiguous amino acids are typically retained upon protein denaturing, whereas epitopes formed by tertiary folding are typically lost upon protein denaturing. An epitope typically includes at least 3, and more usually, at least 5 or 8-10 amino acids in a unique spatial conformation.

That an antibody “specifically binds” to an epitope or protein means that the antibody reacts or associates more frequently, more rapidly, with greater duration, with greater affinity, or with some combination of the above to an epitope or protein than with alternative substances, including unrelated proteins. In certain embodiments, “specifically binds” means, for instance, that an antibody binds to a protein with a K_{D} of about 0.1 mM or less, but more usually less than about 1 μM.

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cell or compositions include those which have been purified to a degree that they are no longer in a form in which they are found in nature. In some embodiments, an antibody, polynucleotide, vector, cell, or composition which is isolated is substantially pure.

As used herein, “substantially pure” refers to material which is at least 50%, 60%, 70%, 80%, 90%, 95%, or 99% pure (i.e., free from contaminants).

A “variable region” of an antibody refers to the variable region of the antibody light chain or the variable region of the antibody heavy chain, either alone or in combination. The variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen-binding site of antibodies. Techniques for determining CDRs are well known in the art, and include: (1) an approach based on cross-species sequence variability (see Kabat et al., Sequences of Proteins of Immunological Interest, (5th ed., 1991, National Institutes of Health, Bethesda Md.)); and (2) an approach based on crystallographic studies of antigen-antibody complexes (Al-lazikani et al., J Molec Biol 273:927-948 ((1997))). In addition, combinations of these two approaches are sometimes used in the art to determine CDRs.

The term “antigen” or “immunogen” as used herein refers to any substance capable of eliciting an immune response when introduced into a subject. An immune response, includes for example, the formation of antibodies and/or cell-mediated immunity. Exemplary antigens include, but are not limited to, infectious agents (e.g., bacteria, viruses, fungi, prion, parasite, and the like), polypeptides (e.g., proteins, including viral proteins), polynucleotides (e.g., DNA, RNA), cells, and compositions containing antigens or immunogens.

The term “vector” means a construct, which is capable of delivering, and preferably expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

The terms “polypeptide,” “peptide,” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this invention are based upon antibodies, in certain embodiments, the polypeptides can occur as single chains or associated chains.

The term “cell” is understood to mean embryonic, fetal, pediatric, or adult cells or tissues, including but not limited to, stem cells, precursors cells, and progenitor cells. Examples of cells include but are not limited to immune cell (e.g., T cells, macrophages, dendritic cells), stem cell, progenitor cell, islet cell, bone marrow cells, hematopoietic cells, tumor cells, lymphocytes, leukocytes, granulocytes, hepatocytes, monocytes, macrophages, fibroblasts, neural cells, mesenchymal stem cells, neural stem cells, or other cells with regenerative properties and combinations thereof. A cell can also refer to cells from a microorganism, including, but not limited to, a bacterial cell, a yeast cell, or a fungal cell.

“Infected cells,” as used herein, includes cells infected naturally by viral entry into the cell, or transfection of the cells with viral genetic material through artificial means. These methods include, but are not limited to, calcium phosphate transfection, DEAE-dextran mediated transfection, microinjection, lipid-mediated transfection, electroporation, or infection.

By “viral host cell” is meant a cell having or is permissive for viral infection. For HIV, exemplary viral host cells include macrophages, dendritic cells, and T cells.

The term “subject” or “patient” refers to an animal which is the object of treatment, observation, or experiment. By way of example only, a subject includes, but is not limited to, a mammal, including, but not limited to, a human or a non-human mammal, such as a non-human primate, murine, bovine, equine, canine, ovine, or feline.

“Administering” is defined herein as a means of providing an agent to a subject in a manner that results in the agent being inside the subject's body. Such an administration can be by any route including, without limitation, oral, transdermal, mucosal (e.g., vagina, rectum, oral, or nasal mucosa), by injection (e.g., subcutaneous, intravenous, parenterally, intraperitoneally, intrathecal), or by inhalation (e.g., oral or nasal). Pharmaceutical preparations are, of course, given by forms suitable for each administration route.

By “agent” is meant any small molecule chemical compound, antibody, nucleic acid molecule, or polypeptide, or fragments thereof. In embodiments, the agent is a VHH.

As used herein, the terms “prevent,” “preventing,” “prevention,” “prophylactic treatment,” and the like, refer to reducing the probability of developing a disease or condition in a subject, who does not have, but is at risk of or susceptible to developing a disease or condition, e.g., viral infection.

As used herein, the terms “treat,” treating,” “treatment,” and the like refer to reducing or ameliorating a disorder or condition, e.g., viral infection, and/or symptoms associated therewith. It will be appreciated that, although not precluded, treating a disorder or condition does not require that the disorder, condition, or symptoms associated therewith be completely eliminated.

The term “therapeutic effect” refers to some extent of relief of one or more of the symptoms of a disorder or condition, e.g., viral infection. In reference to the treatment of HIV, a therapeutic effect refers to one or more of the following: 1) reduction in the number of infected cells; 2) reduction in the concentration of virions present in serum; 3) inhibiting (e.g., slowing to some extent, preferably stopping) the rate of HIV replication; 4) increasing T-cell count; 5) relieving or reducing to some extent one or more of the symptoms associated with HIV; and 6) relieving or reducing the side effects associated with the administration of anti-retroviral agents.

“Therapeutically effective amount” is intended to qualify the amount required to achieve a therapeutic effect. A physician or veterinarian having ordinary skill in the art can readily determine and prescribe the “therapeutically effective amount” of an agent of the present invention, e.g., VHH. For example, the physician or veterinarian could start doses of the agent(s) of the invention employed in a pharmaceutical composition at levels lower than that required in order to achieve the desired therapeutic effect and gradually increase the dosage until the desired effect is achieved.

As used herein, terms such as “inhibiting viral transmission” means any interference in, inhibition of, and/or prevention of viral infection.

“Pharmaceutically acceptable” refers to approved or approvable by a regulatory agency of the Federal or a state government or listed in the U.S. Pharmacopeia or other generally recognized pharmacopeia for use in animals, including humans.

“Pharmaceutically acceptable excipient, carrier or adjuvant” refers to an excipient, carrier or adjuvant that can be administered to a subject, together with a labeled antigen, and which does not destroy the pharmacological activity thereof and is nontoxic when administered in doses sufficient to deliver a therapeutic amount of the VHH.

As used herein, the term “homology” is defined as the percentage of residues in the candidate amino acid sequence that are identical with the residues in the amino acid sequence of their native counterparts after aligning the sequences and introducing gaps, if necessary, to achieve the maximum percent homology by known methods (e.g., BLAST alignment tools). Methods and computer programs for the alignment are well-known in the art.

As known in the art, “sequence identity” between two polypeptides or two polynucleotides is determined by comparing the amino acid or nucleic acid sequence of one polypeptide or polynucleotide to the sequence of a second polypeptide or polynucleotide. When discussed herein, whether any particular polypeptide is at least about 40%, 45%, 50%, 55%, 60%, 65%, 70%, 75%, 80%, 85%, 90% or 95% identical to another polypeptide can be determined using methods and computer programs/software known in the art such as, but not limited to, the BESTFIT program (Wisconsin Sequence Analysis Package, Madison, Wis.). BESTFIT uses the local homology algorithm of Smith et al., Adv Appl Math 2:482-489 (1981), to find the best segment of homology between two sequences. When using BESTFIT or any other sequence alignment program to determine whether a particular sequence is, for example, 95% identical to a reference sequence according to the present invention, the parameters are set, of course, such that the percentage of identity is calculated over the full length of the reference polypeptide sequence and that gaps in homology of up to 5% of the total number of amino acids in the reference sequence are allowed.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any agents, compositions, or methods provided herein can be combined with one or more of any of the other compositions and methods provided herein.

### DESCRIPTION OF THE DRAWINGS

FIG. 1 includes a schematic of the transwell system used to study movement of free or cell-associated virus across an epithelial barrier.

FIG. 2 includes a graph showing that monocyte-associated HIV-1_{Ba-L} crosses a human cervical epithelial cell barrier more efficiently than PB L-associated HIV-1_{Ba-L} or a cell-free virus. Data are expressed as mean±SD of HIV-1 p24 Ag (pg/ml) in the basal side supernatant fluid from the transwell culture, with (w/) and without (w/o) ME-180 cervical cells plated on the insert membrane. A representative experiment (n=3) is shown (p<0.05 for comparisons between all inoculum types with and without ME-180 cells, and p<0.05 for comparisons between inoculum types with ME-180 cells).

FIG. 3 includes a graph showing that anti-ICAM-1 antibodies block the transmission of monocyte-associated HIV-1. Culturing HIV-1-infected monocytes with anti-ICAM-1 antibodies (20 ug/ml; HA58 (IgG1)) inhibits the transepithelia ltransmission of cell-associated HIV-1 across a ME-180 monolayer. Anti-E-cadherin (20 ug/ml; 67A4 (IgG1) and E.4.6 (IgG1)), anti-CD103 (20 ug/ml; 2G5 (IgG2a)), murine IgG1 (mIgG1; 20 ug/ml), and murine IgG2a (mIgG2a; 20 ug/ml) antibodies added with HIV-1-infected monocytes to the transwell cultures have no effect on the transepithelial transmission of cell-associated HIV-1. Results are expressed as mean±SD HIV-1 p24 concentrations from the basal side supernatant fluid and are representative of three separate experiments. p<0.05.

FIG. 4 includes a schematic representation of the in vivo Hu-PBL-SCID vaginal transmission challenge model.

FIG. 5 includes graphs showing that there is a synergistic interaction between anti-ICAM-1 and anti-CD18 Mab. 1×10⁶ HIV-1 infected PBMC were added with designated antibodies or 50:50 mix at 10, 20, or 50 mg/ml to apical side of HT-3 monolayers grown on permeable transwell supports and allowed to transmigrate for 24 hours. Error bars represent +/−1 standard deviation.

FIG. 6 includes histology stains showing the lack of toxicity associated with sustained administration of anti-ICAM-1.

FIG. 7 includes a graph showing that anti-ICAM Fab blocks transmission of HIV-1-infected PBMC across an HT-3 cell monolayer. HIV-1-infected PBMCs (1×10⁶) were added with the designated treatment to the apical side of HT-3 monolayers grown on permeable transwell supports, and allowed to transmigrate for 24 hours. All intact antibodies were used at a concentration of 100 μg/ml, and all Fabs were used at 67 μg/ml to equalize the available binding sites. Data are expressed as mean±SD basilar HIV-1 p24 concentration or viable PBMCs counted. *, p<0.05; **, p<0.01.

FIG. 8 includes a graph showing that antibodies to ICAM-1 and CD18 do not reduce transmission of cell-free HIV-1 across a cervical epithelial monolayer. 20 μg/ml total of designated antibody or mixture was added to 1×10³ TCID50 HIV-1 JR-CSF immediately before addition to the apical side of an ME-180 transwell culture with 1×10⁶ PHA blasts in the basal compartment, and incubated for 24 hours at 37° C. Transmission was measured by HIV-1 p24 ELISA on basal supernatants. Data are expressed as mean of triplicate wells ±SD.

FIG. 9 includes a graph showing that antibodies to ICAM-1 and CD18, both alone and in combination, reduce infection of target cells beneath the cervical epithelium. 20 μg/ml total of designated antibody or mixture was added to 1×10³ TCID₅₀ HIV-1 JR-CSF immediately before addition to the apical side of an ME-180 transwell culture with 1×10⁶ PHA blasts in the basal compartment and incubated for 24 h at 37° C. Transwells were then removed, and PBMC supernatants were sampled at 48 h intervals. Transmission was measured by HIV-1 p24 ELISA on basal supernatants. Data are expressed as mean of triplicate wells ±SD. Open symbols indicate significant difference (p<0.05) from untreated cells.

FIG. 10 includes histology stains showing that CD11c+ dendritic cells are present in reconstituted mice. Human fetal thymus and liver (18-24 weeks gestation, Advanced Bioscience Resources, Kensington, Md.) were surgically implanted under the kidney capsule into 6- to 8-week-old nonobese diabetic-severe combined immunodeficient (NOD/LtSz-Prkdcoscid4) mice (NOD/SCID) (Jackson Laboratories, Bar Harbor, Me.). CD34+ cells from autologous fetal liver tissue were isolated and then immediately frozen (−80° C.) and stored in liquid nitrogen until transplantation. Three weeks after implantation, the mice were sublethally irradiated (325 cGy from a 137Cs gamma radiation source) and transplanted intravenously within 24 hours with 0.2-2.5 10⁶ CD34+ cells. The dark staining areas represent cells expressing hCD11c.

FIG. 11 includes a map of the cloning vector used for expression of the human ICAM-1 single-chain antibody. The vector allows for direct cloning of PCR-amplified fragments of the variable domains of the H and L chains.

FIGS. 12A-12D include flow cytometry images showing that Anti-ICAM-1 scFvs produced by lactobacilli specifically bind ICAM-expressing cells. CHO cells overexpressing ICAM-1 were incubated with intact anti-ICAM MT-M5 (blue), negative (neg) control antibody (red), a 1/10 dilution of lactobacillus culture supernatant (A), or dilutions of purified scFv from a 20 μg/ml stock (green) (B-D). scFv binding was detected using a mouse anti-E tag followed by FITC-labeled goat anti-mouse.

FIG. 13 includes a graph showing that anti-ICAM scFvs (67 ug/ml) block transmission of HIV-1 p24 across an HT-3 cell monolayer. HIV-1-infected PBMCs (1×10⁶) were added with the designated treatment to the apical side of HT-3 monolayers grown on permeable transwell supports, and allowed to transmigrate for 24 hours. Data are expressed as mean±SD basilar HIV-1 p24 concentrations or viable PBMC control. *, p<0.05; p<0.01.

FIG. 14 includes a map of the Lactobacillus vector for surface-anchored expression of variable domain of llama heavy-chain (VHH1). The corresponding VHH1-secreted construct was equipped with a TAA stop codon, inserted after the E-tag sequence (the arrow indicates the stop codon). Ampr, ampicillin resistance gene; deleted Tldh, remaining sequence after the deletion of the transcription terminator of the ldh gene of L. casei; Ery, erythromycin resistance gene; long anchor, anchor sequence from the proteinase P gene of L. casei (244 aa); N-terminus PrtP, N-terminal 36 aa of the PrtP gene; Ori+, origin of replication of E. coli; Ori, origin of replication of Lactobacillus; Pldh, promoter sequence of the lactate dehydrogenase (ldh) gene of L. casei; Rep, repA gene of plasmid p353-2 from L. pentosis; SS prtP, signal sequence of the PrtP gene (33 aa); Tcbh, transcription terminator sequence of the conjugated bile acid hydrolase gene of L. plantarum 80.-terminus PrtP, 36aa of the PrtP gene; Ori+, origin of replication of E. coli; Ori, origin o freplication of Lactobacillus; Pldh, promoter sequence of the lactate dehydrogenase (ldh) gene of L. casei; Rep, repA gene of plasmid p353-2 from L. pentosis; SS prtP, signal sequence of the PrtP gene (33 aa); Tcbh, transcription terminator sequence of the conjugated bile acid hydrolase gene of L. plantarum 80.

FIG. 15 includes a graph showing the results from the ELISA analysis of anti-CD18 production in Alpaca. Alpacas were immunized with recombinant purified CD18. A 96 well plate was coated with CD18, and alpaca sera was extracted and diluted onto the bound antigen. HRP conjugated goat-anti-llama (cross-reactive with alpaca) secondary antibodies were added to bound antibodies from the sera. ABTS substrate was added, and the plate was read at 405 nm Absorbance readings were adjusted to negative background readings.

FIG. 16 includes a graph demonstrating the ability of serum from CD18-immunized alpaca to block cell-associated HEV-1 transmission in transwell assay. 2×10⁵ HIV-1_{Ba-L} infected macrophages were placed in the upper chamber of a transwell with chambers separated by a confluent layer of MT-4 cervical epithelial cells. The upper chamber also contained either the indicated dilutions of serum from an alpaca immunized with the recombinant ectodomain of CD18 or anti-CD18 monoclonal antibody H52 at a concentration of 20 ug/ml. The vertical axis indicates p24 recovered from the lower chamber after 24 hours.

FIG. 17 includes a schematic representation of a construct used for expression of VHH by lactobacilli.

FIGS. 18A-18C show that an anti-CD18 alpaca derived phage library can be used to successfully identify antigen specific VHH polypeptides. FIG. 18A includes a graph showing the six anti-CD18 clones that were identified during the panning experiments. Of the six phage clones, two were unique (VHH21 and VHH-173). FIG. 18B includes a graph showing the binding of clone VHH21 to GST-CD18. FIG. 18C includes a graph showing the binding of clone VHH73 to GST-CD18.

FIGS. 19A and 19B provide the nucleic acid (FIG. 19A; SEQ ID NO:1) and amino acid (FIG. 19B; SEQ ID NO:2) sequences of the VHH21 clone.

FIGS. 20A and 20B provide the nucleic acid (FIG. 20A; SEQ ID NO:3) and amino acid (FIG. 20B; SEQ ID NO:4) sequences of the VHH73 clone.

FIGS. 21A-21C show that llamas immunized with herpes simplex virus type-2 glycoprotein D antigen produce HSV-2 neutralizing antibodies. FIG. 21A includes a table showing the neutralizing capability of serum obtained from a non-immunized llama. FIG. 21B includes a table showing the neutralizing capability of serum obtained from an immunized alpaca. FIG. 21C includes a graph comparing the HSV-2 neutralizing ability of sera obtained from immunized versus non-immunized llamas.

### DETAILED DESCRIPTION OF THE INVENTION

The invention features novel compositions, methods, and kits for preventing or inhibiting viral infection (e.g., HIV).

HIV infections are acquired most often through sexual contact, with the majority of sexual transmission of HIV worldwide occurring as a result of heterosexual contact. Women of childbearing age are at the greatest risk for HIV infection, which has resulted in a corresponding increase in HIV infection of women, newborns and infants worldwide. As such, much efforts have been devoted to developing microbicides, which are a potentially woman-controlled preventive intervention that may be used to reduce the incidence of new HIV infections.

The difficulty in eliciting protective responses at the site of exposure and the genetic diversity of candidate target antigens of HIV-1 have reinforced interest in preventive measures that do not depend on either the elicitation of immune responses or the targeting of viral antigens. The paragon of such approaches, of course, is the condom, but the persistent expansion of the HIV-1 epidemic despite their availability and aggressive education programs their use is testimony to their lack of acceptance (Baleta, Lancet 353:653 (1999)). Resistance to their use stems, among other reasons, from the impression that sex without condoms is more exciting and the sentiment that demanding use of condoms suggests mistrust in a partner (Chirwa, Network 13:31-32 (1993)). Additionally, it is impossible to separate the disease prevention function of condoms from their contraceptive function, the latter of which may not always be desired. While microbicides clearly have the potential to function with or without contraceptive activity, current microbicides still fail to address many of the condom-related issues. For example, their potential to provide “excessive” lubrication might also make sex “less exciting”, and their use would still be apparent, raising issues of trust.

Because lactobacilli are already the major microbial component of the vaginal environment, their use as a microbicide delivery vehicle would have none of the issues associated with more traditional microbicides: their presence would be transparent to users and they would therefore not alter the sexual experience. The persistence of engineered lactobacilli within the vagina over a period of weeks to months would not require coitally-associated intervention and, once the desired antibodies are integrated into the bacterial genome in a stable manner, they would be inexpensive to produce and, as either freeze-dried or spray-dried bacteria could maintain viability for extended periods (Corcoran et al., Appl Environ Microbiol 72:5104-5107 (2006)).

The issue that arises with all antibody-based anti-HIV-1 technologies is what antigens to target. Accordingly, the invention is based, at least in part, on the discovery that ICAM-1 and CD18 antibodies are effective at inhibiting HIV transmission across the epithelium. The invention provides camelid-derived antibodies or fragments that specifically bind to ICAM-1 or CD18. The invention also provides recombinant microorganisms (e.g., bacteria) expressing the camelid-derived antibodies, as well as their use in preventing HIV infection. The invention further provides compositions (e.g., microbicides) comprising the recombinant microorganism, and their use for epithelial (e.g., vaginal and anal) delivery.

### Camelid Derived Antibodies

The present invention relates to camelid-derived antibodies, e.g., variable heavy chain polypeptides (VHH), that specifically bind to ICAM-1, CD18, herpes simplex virus-2 (HSV-2) glycoprotein D, or fragments thereof.

a. ICAM-1

ICAM-1 is a single-chain glycoprotein adhesion molecule constitutively expressed on resting endothelial cells, resting monocytes, resting epithelial cells as well as activated T-cells. ICAM-1 expression is induced by a variety of cytokines including IFN-γ, TNFα, and IL-1. The CD18 family of cellular adhesion molecules mediate interactions between cells of the immune and inflammatory system. LFA-1, also known as Lymphocyte Function-Associated Antigen-1 or CD11a/CD18, recognizes and binds to ICAM-1, ICAM-2 and ICAM-3 on the endothelium. The heterodimeric structure of LFA-1 consists of an alpha and a beta chain. As a member of the integrin family, LFA-1 plays a role in many cellular processes such as migration, antigen presentation, and cell proliferation. For example, LFA-1 mediates the binding of leukocytes to endothelial cells permitting the migration of leukocytes from the bloodstream into the tissue. ICAM-1 is the primary ligand for LFA-1. ICAM-1 is anchored to the endothelium by a transmembrane domain, has a short cytoplasmic tail, contains five extracellular immunoglobulin-like domains, and is expressed on HIV-infected monocytes and epithelial cells.

The sequence of ICAM-1 is well-known in the art. For example, a representative nucleic acid sequence for ICAM-1 can be found at GenBank Accession No. X06990.1, which is reproduced below:

CTCAGCCTCGCTATGGCTCCCAGCAGCCCCCGGCCCGCGCTGCCCGCACTCCTGGTCCTGC
TCGGGGCTCTGTTCCCAGGACCTGGCAATGCCCAGACATCTGTGTCCCCCTCAAAAGTCAT
CCTGCCCCGGGGAGGCTCCGTGCTGGTGACATGCAGCACCTCCTGTGACCAGCCCAAGTT
GTTGGGCATAGAGACCCCGTTGCCTAAAAAGGAGTTGCTCCTGCCTGGGAACAACCGGAA
GGTGTATGAACTGAGCAATGTGCAAGAAGATAGCCAACCAATGTGCTATTCAAACTGCCC
TGATGGGCAGTCAACAGCTAAAACCTTCCTCACCGTGTACTGGACTCCAGAACGGGTGGA
ACTGGCACCCCTCCCCTCTTGGCAGCCAGTGGGCAAGAACCTTACCCTACGCTGCCAGGTG
GAGGGTGGGGCACCCCGGGCCAACCTCACCGTGGTGCTGCTCCGTGGGGAGAAGGAGCTG
AAACGGGAGCCAGCTGTGGGGGAGCCCGCTGAGGTCACGACCACGGTGCTGGTGAGGAG
AGATCACCATGGAGCCAATTTCTCGTGCCGCACTGAACTGGACCTGCGGCCCCAAGGGCT
GGAGCTGTTTGAGAACACCTCGGCCCCCTACCAGCTCCAGACCTTTGTCCTGCCAGCGACT
CCCCCACAACTTGTCAGCCCCCGGGTCCTAGAGGTGGACACGCAGGGGACCGTGGTCTGT
TCCCTGGACGGGCTGTTCCCAGTCTCGGAGGCCCAGGTCCACCTGGCACTGGGGGACCAG
AGGTTGAACCCCACAGTCACCTATGGCAACGACTCCTTCTCGGCCAAGGCCTCAGTCAGT
GTGACCGCAGAGGACGAGGGCACCCAGCGGCTGACGTGTGCAGTAATACTGGGGAACCA
GAGCCAGGAGACACTGCAGACAGTGACCATCTACAGCTTTCCGGCGCCCAACGTGATTCT
GACGAAGCCAGAGGTCTCAGAAGGGACCGAGGTGACAGTGAAGTGTGAGGCCCACCCTA
GAGCCAAGGTGACGCTGAATGGGGTTCCAGCCCAGCCACTGGGCCCGAGGGCCCAGCTCC
TGCTGAAGGCCACCCCAGAGGACAACGGGCGCAGCTTCTCCTGCTCTGCAACCCTGGAGG
TGGCCGGCCAGCTTATACACAAGAACCAGACCCGGGAGCTTCGTGTCCTGTATGGCCCCC
GACTGGACGAGAGGGATTGTCCGGGAAACTGGACGTGGCCAGAAAATTCCCAGCAGACT
CCAATGTGCCAGGCTTGGGGGAACCCATTGCCCGAGCTCAAGTGTCTAAAGGATGGCACT
TTCCCACTGCCCATCGGGGAATCAGTGACTGTCACTCGAGATCTTGAGGGCACCTACCTCT
GTCGGGCCAGGAGCACTCAAGGGGAGGTCACCCGCGAGGTGACCGTGAATGTGCTCTCCC
CCCGGTATGAGATTGTCATCATCACTGTGGTAGCAGCCGCAGTCATAATGGGCACTGCAG
GCCTCAGCACGTACCTCTATAACCGCCAGCGGAAGATCAAGAAATACAGACTACAACAGG
CCCAAAAAGGGACCCCCATGAAACCGAACACACAAGCCACGCCTCCCTGAACCTATCCCG
GGACAGGGCCTCTTCCTCGGCCTTCCCATATTGGTGGCAGTGGTGCCACACTGAACAGAGT
GGAAGACATATGCCATGCAGCTACACCTACCGGCCCTGGGACGCCGGAGGACAGGGCATT
GTCCTCAGTCAGATACAACAGCATTTGGGGCCATGGTACCTGCACACCTAAAACACTAGG
CCACGCATCTGATCTGTAGTCACATGACTAAGCCAAGAGGAAGG

The corresponding amino acid sequence is as follows:

MAPSSPRPALPALLVLLGALFPGPGNAQTSVSPSKVILPRGGSVLVTCSTSCDQPKLLGIETPLP
KKELLLPGNNRKVYELSNVQEDSQPMCYSNCPDGQSTAKTFLTVYWTPERVELAPLPSWQPV
GKNLTLRCQVEGGAPRANLTVVLLRGEKELKREPAVGEPAEVTTTVLVRRDHHGANFSCRTE
LDLRPQGLELFENTSAPYQLQTFVLPATPPQLVSPRVLEVDTQGTVVCSLDGLFPVSEAQVHLA
LGDQRLNPTVTYGNDSFSAKASVSVTAEDEGTQRLTCAVILGNQSQETLQTVTIYSFPAPNVIL
TKPEVSEGTEVTVKCEAHPRAKVTLNGVPAQPLGPRAQLLLKATPEDNGRSFSCSATLEVAGQ
LIHKNQTRELRVLYGPRLDERDCPGNWTWPENSQQTPMCQAWGNPLPELKCLKDGTFPLPIG
ESVTVTRDLEGTYLCRARSTQGEVTREVTVNVLSPRYEIVIITVVAAAVIMGTAGLSTYLYNRQ
RKIKKYRLQQAQKGTPMKPNTQATPP

Accordingly, those of ordinary skill in the art will readily know how to make and use the camelid-derived ICAM-1 antibodies of the present invention based the teachings in the application.

b. CD18

CD18 is the beta-chain component of the CD11a/CD18 heterodimer that is LFA-1. As discussed above, CD18/LFA-1 interacts with ICAM-1 on monocytes.

The sequence of CD18 is well-known in the art. For example, a representative nucleic acid sequence for CD18 is reproduced below:

CTGCGACCAGGCCAGGCAGCAGCGTTCAACGTGACCTTCCGGCGGGCCAAGGGCTACCCC
ATCGACCTGTACTATCTGATGGACCTCTCCTACTCCATGCTTGATGACCTCAGGAATGTCA
AGAAGCTAGGTGGCGACCTGCTCCGGGCCCTCAACGAGATCACCGAGTCCGGCCGCATTG
GCTTCGGGTCCTTCGTGGACAAGACCGTGCTGCCGTTCGTGAACACGCACCCTGATAAGCT
GCGAAACCCATGCCCCAACAAGGAGAAAGAGTGCCAGCCCCCGTTTGCCTTCAGGCACGT
GCTGAAGCTGACCAACAACTCCAACCAGTTTCAGACCGAGGTCGGGAAGCAGCTGATTTC
CGGAAACCTGGATGCACCCGAGGGTGGGCTGGACGCCATGATGCAGGTCGCCGCCTGCCC
GGAGGAAATCGGCTGGCGCAACGTCACGCGGCTGCTGGTGTTTGCCACTGATGACGGCTT
CCATTTCGCGGGCGACGGAAAGCTGGGCGCCATCCTGACCCCCAACGACGGCCGCTGTCA
CCTGGAGGACAACTTGTACAAGAGGAGCAACGAATTCGACTACCCATCGGTGGGCCAGCT
GGCGCACAAGCTGGCTGAAAACAACATCCAGCCCATCTTCGCGGTGACCAGTAGGATGGT
GAAGACCTACGAGAAACTCACCGAG

The corresponding amino acid sequence is as follows:

LRPGQAAAFNVTFRRAKGYPIDLYYLMDLSYSMLDDLRNVKKLGGDLLRALNEITESGRIGFG
SFVDKTVLPFVNTHPDKLRNPCPNKEKECQPPFAFRHVLKLTNNSNQFQTEVGKQLISGNLDA
PEGGLDAMMQVAACPEEIGWRNVTRLLVFATDDGFHFAGDGKLGAILTPNDGRCHLEDNLY
KRSNEFDYPSVGQLAHKLAENNIQPIFAVTSRMVKTYEKLTE

Accordingly, those of ordinary skill in the art will readily know how to make and use the camelid-derived CD18 antibodies of the present invention based the teachings in the application.

c. HSV-2 Glycoprotein D

A clear advantage of the recombinant microorganism-based VHH delivery system is that once it has been developed, the technology can be modified to target a variety of different sexually-transmitted diseases. For example, more than 30 epidemiologic studies have demonstrated that prevalent herpes simplex virus type-2 (HSV-2) infection is associated with a 2-4 fold enhanced risk of HIV-1 acquisition (Baeten et al., Aids 21:1771-1777 (2007); Brown et al., Aids 21:1515-1523 (2007); and Corey et al., J Acquir Immune Defic Syndr 35:435-445 (2004)). As such, the invention includes VHH specific for HSV-2.

Herpesviruses are enveloped double stranded DNA-containing viruses in an icosahedral nucleocapsid. HSV-2 is associated with human genital herpes. While glycoprotein-based vaccines against HSV-2 infection have shown only moderate efficacy in humans (Corey et al., Jama 282:331-340 (1999); and Milligan et al., Sex Transm Dis 29:597-605 (2002)), evidence from guinea pig models of the disease indicate that passive immunization can significantly ameliorate the course of the disease (Milligan et al.). Recent evidence that substantial Ab-mediated protection against genital HSV-2 disease can be achieved by either Fc-gammaR-dependent or -independent mechanisms provides a basis for pursuing technologies for in situ secretion of VHH (Chu et al., J Reprod Immunol 78:58-67 (2008)), and the presence of a CD4⁺ CTL epitope in HSV-2 glycoprotein D makes this protein a leading candidate as a target for HSV-2 treatment (Cooper et al., Cell Immunol 2:113-120 (2006)).

The sequence of HSV-2 glycoprotein D is well-known in the art. For example, a representative nucleic acid sequence for HSV-2 glycoprotein D is reproduced below:

AAATACGCCTTAGCAGACCCCTCGCTTAAGATGGCCGATCCCAATCGAT
TTCGCGGGAAGAACCTTCCGGTTTTGGACCAGCTGACCGACCCCCCCGG
GGTGAAGCGTGTTTACCACATTCAGCCGAGCCTGGAGGACCCGTTCCAG
CCCCCCAGCATCCCGATCACTGTGTACTACGCAGTGCTGGAACGTGCCT
GCCGCAGCGTGCTCCTACATGCCCCATCGGAGGCCCCCCAGATCGTGCG
CGGGGCTTCGGACGAGGCCCGAAAGCACACGTACAACCTGACCATCGCC
TGGTATCGCATGGGAGACAATTGCGCTATCCCCATCACGGTTATGGAAT
ACACCGAGTGCCCCTACAACAAGTCGTTGGGGGTCTGCCCCATCCGAAC
GCAGCCCCGCTGGAGCTACTATGACAGCTTTAGCGCCGTCAGCGAGGAT
AACCTGGGATTCCTGATGCACGCCCCCGCCTTCGAGACCGCGGGTACGT
ACCTGCGGCTAGTGAAGATAAACGACTGGACGGAGATCACACAATTTAT
CCTGGAGCACCGGGCCCGCGCCTCCTGCAAGTACGCTCTCCCCCTGCGC
ATCCCCCCGGCAGCGTGCCTCACCTCGAAGGCCTACCAACAGGGCGTGA
CGGTCGACAGCATCGGGATGCTACCCCGCTTTATCCCCGAAAACCAGCG
CACCGTCGCCCTATACAGCTTAAAAATCGCCGGGTGGCACGGCCCCAAG
CCCCCGTACACCAGCACCCTGCTGCCGCCGGAGCTGTCCGACACCACCA
ACGCCACGCAACCCGAACTCGTTCCGGAAGACCCCGAGGACTCGGCCCT
CTTAGAGGATCCCGCCGGGACGGTGTCTTCGCAGATCCCCCCAAACTGG
CACATCCCGTCGATCCAGGACGTCGCGCCGCACCACGCCCCCGCCGCCC
CCAGCAACCCG

The corresponding amino acid sequence is as follows:

KYALADPSLKMADPNRFRGKNLPVLDQLTDPPGVKRVYHIQPSLEDPFQ
PPSIPITVYYAVLERACRSVLLHAPSEAPQIVRGASDEARKHTYNLTIA
WYRMGDNCAIPITVMEYTECPYNKSLGVCPIRTQPRWSYYDSFSAVSED
NLGFLMHAPAFETAGTYLRLVKINDWTEITQFILEHRARASCKYALPLR
IPPAACLTSKAYQQGVTVDSIGMLPRFIPENQRTVALYSLKIAGWHGPK
PPYTSTLLPPELSDTTNATQPELVPEDPEDSALLEDPAGTVSSQIPPNW
HIPSIQDVAPHHAPAAPSNP

Accordingly, those of ordinary skill in the art will readily know how to make and use the camelid-derived HSV-2 glycoprotein D antibodies of the present invention based the teachings in the application.

d. VHH Polypeptides

The classical mammalian antibody is too large and too complex a molecule to be readily produced in bacteria. Initial efforts to develop a lactobacillus-based in situ expression system used single chain variable region antibodies, structures created by fusing DNA encoding the light and heavy chain variable regions of an antibody with a known specificity to a linker that ensures folding of the single chain such that the V_{H} and V_{L} come together to form a functional Fab-like structure. The variable region domains are obtained by reverse transcription of Ig mRNA from hybridomas producing antibodies of the desired specificity, followed by amplification using primers specific for the variable region of mouse IgG, as described in Froyen et al., Mol Immunol 30:805-812 (1993); Ward, Adv Pharmacol 24:1-20 (1993); and Seegers, Trends Biotechnol 20:508-515 (2002). However, as described in detail below, variable results are achieved with this approach. Lactobacilli are able to secrete antibody to ICAM-1 at concentrations in culture of up to 5 ug/ml, well within the range of efficacy observed in in vitro assays. On the other hand, the levels of secretion of antibody to CD18 never exceeds 1 ug/ml. Higher concentrations of the anti-CD18 antibody are produced, but are retained within the bacteria and not secreted. Although not wishing to be bound by any theory, it is thought that the basis for the differences in secretion between some scFv and others is not well understood and is likely related to protein folding differences, although as will be indicated, the size of these molecules, while much smaller than antibodies, may still be problematic. Because the scientific basis for success or failure in obtaining high levels of secretion is not well understood, there are limited maneuvers that can be undertaken to attempt to enhance secretion of scFv which, independent of the bacterial expression system, typically has a success rate of approximately 50%.

In recent years, a newer technology has become available that markedly enhances the success rate of obtaining effective levels of bacterially secreted antibodies. This advance stems from the observation that approximately 50% of the antibodies produced by members of the camelid family contain no light chain and that a single domain within the N terminus of the heavy chain molecule acts as the binding domain for antigen (Muyldermans et al., J Mol Recognit 12:131-140 (1999)). The amino acid sequence of the variable domain of the naturally occurring heavy-chain antibodies reveals the necessary adaptations to compensate for the absence of the light chain. The presence of the heavy-chain antibodies and the possibility of immunizing camelids allows for the production of antigen binders consisting of a single domain only. These minimal antigen-binding fragments are well expressed in bacteria (Pant et al., J. Infect Dis 194:1580-1588 (2006)), bind the antigen with affinity in the nM range and are very stable. The structure of the camelid single domain, termed VHH or nanobodies because of their relatively small size compared to Fab, is homologous to the human variable heavy chain (VH). However, the antigen-binding loop structures deviate fundamentally from the canonical structures described for human or mouse VHs (Muyldermans et al.; Harmsen et al., Appl Microbiol Biotechnol 77:13-22 (2007); and Alvarez-Rueda et al., Mol Immunol 44:1680-1690 (2007)).

Contrary to conventional antibodies, VHHs have been shown to remain functional at 90° C. or after incubation at high temperatures (Harmsen et al.). This high apparent stability is mainly attributed to their efficient refolding after chemical or thermal denaturation and to a lesser extent because of an increased resistance to denaturation (Hatinsen et al.). Unlike the situation with scFv, high levels of secretion of VHH are routinely obtained following their transfection into bacteria (Pant et al.). Of particular importance, at least two groups have examined the immunogenicity of VHH in mouse systems and have found that multiple injections of VHHs have not shown any immunogenicity as assessed by the presence of specific antibodies, T cell proliferation, or cytokine levels (Cortez-Retamozo et al., Cancer Res 64:2853-2857 (2004); and Coppieters et al., Arthritis Rheum 54:1856-1866 (2006)). This could be attributable to their high sequence homology to conventional VH domains and to their high stability, because aggregation of proteins is known to increase immunogenicity (Hermeling et al., Pharm Res 21:897-903 (2004)). Furthermore, this lack of immune response occurred upon systemic exposure to the VHH, not exposure at a site which is, at some level, immunologically “privileged”.

Camelid-derived antibodies and methods for making such antibodies are well-known in the art. See, e.g., U.S. Pat. Nos. 5,759,808; 5,800,988; 5,840,526; 5,874,541; 6,005,079; and 6,015,695, the contents of each of which are incorporated herein by reference in their entirety. As such, those of ordinary skill in the art will readily know how to make and use the camelid-derived antibodies of the present invention.

In embodiments, the camelid-derived antibodies and fragments thereof (e.g., truncated VHH) are obtained using a phage display library. Methods of making and screening phage display libraries are well-known in the art. As such, those of ordinary skill in the art will readily know how to make the phage display libraries described herein and use such libraries to identify camelid-derived antibodies that specifically bind to molecules of interest (e.g., ICAM-1, CD18, and HSV-2 glycoprotein D).

In related embodiments, the peptides in the phage display library are naturally derived VHH and VHH fragments obtained from a camelid (e.g., alpaca or llama) immunized with an antigen of interest (e.g., ICAM-1, CD18, and HSV-2 glycoprotein D).

In other related embodiments, the peptides in the phage display library are synthetic VHH polypeptides, wherein the CDR diversity in the VHH has been introduced by mutagenesis. See U.S. Pat. Appl. Pub. No. 20100330080.

In another related embodiments, the phage display library is a random polypeptide phage library, wherein the library displays at least one peptide having the framework regions of a camelid-derived VHH or an amino acid sequence having at least 60, 70, 80, or 90% sequence identity thereto.

In embodiments, the camelid-derived antibodies and fragments thereof (e.g., truncated VHH) are humanized camelid-derived antibodies or fragments thereof. As with other antibodies of non-human origin, an amino acid sequence of a camelid antibody can be altered recombinantly to obtain a sequence that more closely resembles a human sequence, thereby further reducing the natural low antigenicity of camelid antibodies to humans. Humanized camelid-derived antibodies and methods of making such antibodies are well-known in the art. See, e.g., Vincke et al., J Biol Chem 284:3272-84 (2008), the content of which is hereby incorporated by reference in its entirety. As such, those of ordinary skill in the art will readily know how to make and use the humanized camelid-derived antibodies of the present invention.

### Bacterial Delivery Systems

The invention includes recombinant microorganisms (e.g., natural microflora present in the vagina or rectum) that express at least one of the camelid-derived antibodies described herein (e.g., ICAM-1, CD18, and HSV glycoprotein D). The invention also relates to use of these recombinant microorganisms to express the camelid-derived antibody in situ.

In embodiments, the present invention inhibits transmission of a pathogen (e.g., HIV) by delivering the camelid-derived antibodies to an epithelium of a subject. In related embodiments, the delivery system is a bacterial system, for example, a bacterial species normally present in the flora of the genital tract, oral cavity, and the like. In some embodiments, the bacterial delivery system may be a lactobacillus delivery system, an E. coli delivery system, and the like.

The delivery system may be selected according to the application. For example, a lactobacillus bacterial delivery system may be used for formulating a vaginally applicable microbicide. By way of another example, an E. coli delivery system may be used for formulating a rectally applicable product (such as, e.g., a product to prevent transmission via rectal intercourse). Such delivery systems are known in the art, and those of ordinary skill in the art will readily know how to make and use the recombinant organisms of the present invention. See Kruger et al., Nature Biotech 20:702-706 (2002) and Chancey et al., J Immunol 176:5627-5636 (2002).

For practicing the invention, the camelid-derived antibodies may be in various forms, including, but not limited to, a microbicide, a delayed release delivery system, a solid phase structure, cervical rings, sponges, condoms, gels, creams, suppositories, capsules, and the like. Those of ordinary skill in the art will readily know how to prepare formulations suitable for use in various modes of delivery. For example, protective antibodies may be delivered by systems incorporating a delivery vehicle (e.g., a delayed release delivery system) or in a solid phase structure from which the protective antibodies may be slowly released (e.g., solid phase materials impregnated with protective antibody or fragments, such as cervical rings, gels, creams, sponges, and the like).

Administration of the camelid-derived antibodies may be, preferably, by the consumer himself or herself, including, but not limited to, recombinant microorganisms in a form that can be self-administration by the consumer (e.g., cervical rings, sponges, condoms, gels, creams, suppositories, capsules, and the like). Studies elsewhere have shown certain bacteria to remain viable, without refrigeration, for up to two years in suppository or capsule form; similar viability for bacterial systems according to the present invention may be expected. It will be appreciated that bacteria are not expected to survive forever and therefore replacement may be in order, such as, e.g., every several days (1, 2, 3, 4, 5, 6, 7), weeks (1, 2, 3, 4), or months (1, 2, 3, 4, 5, 6, 7, 8, 9, 10, 11, 12, 18, 24). The invention also provides for the bacteria to also encode a protein useable as part of a detection system to monitor persistence of the bacteria, as an indicator of when a next application of bacteria may be needed.

Advantageously, in the present invention, bacteria may be engineered to produce the camelid-derived antibodies, after which production of the product simply involves growing the bacteria in large quantities and then lyophilizing the bacteria for in situ delivery, which can be done relatively inexpensively. Thus, the invention advantageously provides methods of preventing initial infection and preventing transepithelial viral (e.g., HIV and HSV-2) transmission in which no purification of antibody is necessary and no complex chemical processes are required to synthesize an active product.

### Methods of Treatment

The invention includes methods for inhibiting viral infection. In embodiments, camelid-derived antibodies that specifically bind to ICAM-1, CD18, or HSV-2 glycoprotein D are used to inhibit viral transmission across an epithelium (e.g., a vaginal epithelium, a cervical epithelium, a gastrointestinal epithelium, a rectal epithelium, a colonic epithelium, an oral epithelium).

In one aspect, the methods inhibits the establishment or persistence of viral infection in a subject. In embodiments, the method involves contacting an epithelial cell having or at risk of developing a viral infection with a camelid-derived antibody that specifically binds to ICAM-1, CD18, and/or HSV-2 glycoprotein D. In related embodiments, the methods inhibit transepithelial transmission of the virus.

In one aspect, the methods inhibits viral transmission in a subject. In embodiments, the method involves contacting an epithelial cell having or at risk of developing a viral infection with a camelid-derived antibody that specifically binds to ICAM-1, CD18, and/or HSV-2 glycoprotein D. In related embodiments, the methods inhibit transepithelial transmission of the virus.

In any of the above aspects, the camelid-derived antibody is delivered using any of the bacterial delivery systems described herein. In embodiments, the bacterial delivery system is a lactococcus delivery system. In other embodiments, the bacterial delivery system is an E. coli delivery system.

In any of the above aspects, the virus can be HIV (e.g., HIV-1 or HIV-2) or HSV-2. In embodiments, the virus is cell-associated virus. In other embodiments, the virus is cell-free virus.

Recombinant microorganisms expressing the camelid-derived antibodies of the present invention can be prepared in lyophilized form, which can readily be used in any composition or delivery form. When the recombinant microorganisms are administered to a subject, the recombinant microorganisms will likely be administered as a composition in combination with a pharmaceutically acceptable carrier or excipient. Pharmaceutically acceptable carriers are physiologically acceptable and retain the therapeutic properties of the small molecules, antibodies, nucleic acids, or peptides present in the composition. Pharmaceutically acceptable carriers are well-known in the art and generally described in, for example, Remington's Pharmaceutical Sciences (18^{th} Edition, ed. A. Gennaro, Mack Publishing Co., Easton, Pa., 1990). Suitable dose ranges and cell toxicity levels may be assessed using standard dose range experiments that are well-known in the art. Actual dosages administered may vary depending, for example, on the nature of the disorder, e.g., stage of virus-mediated pathology, the age, weight and health of the individual, as well as other factors.

In embodiments, the recombinant microorganisms are formulated into preparations in solid, semi-solid, liquid or gaseous forms, such as tablets, capsules, pills, powders, granules, dragees, gels, slurries, ointments, solutions, suppositories, injections, inhalants and aerosols. Administration of such formulations can be achieved in various ways, including oral, buccal, parenteral, topical, transdermal, transmucosal, inhalation, nasal, rectal, vaginal, etc., administration. Moreover, recombinant microorganisms can be administered in a local rather than systemic manner, for example, in a depot or sustained release formulation.

For oral administration, the recombinant microorganisms can be readily formulated by combining with pharmaceutically acceptable carriers that are well-known in the art. Such carriers enable the compounds to be formulated as tablets, pills, dragees, capsules, emulsions, lipophilic and hydrophilic suspensions, liquids, gels, syrups, slurries, suspensions, and the like, for oral ingestion by a subject to be treated. Pharmaceutical preparations for oral use can be obtained by mixing the lyophilized recombinant microorganisms with a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; and cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents can be added, such as a cross-linked polyvinyl pyrrolidone, agar, or alginic acid or a salt thereof such as sodium alginate.

Oral recombinant microorganisms formulations can be sustained or extended-release formulations. Methods and ingredients for making sustained or extended-release formulations are well-known in the art. For example, sustained or extended-release formulations can be prepared using natural ingredients, such as minerals, including titanium dioxide, silicon dioxide; zinc oxide, and clay (see U.S. Pat. No. 6,638,521). Exemplified extended release formulations that can be used in delivering recombinant microorganisms include those described in U.S. Pat. Nos. 6,635,680; 6,624,200; 6,613,361; 6,613,358, 6,596,308; 6,589,563; 6,562,375; 6,548,084; 6,541,020; 6,537,579; 6,528,080; and 6,524,621. Controlled release formulations that can be used in delivering recombinant microorganisms include those described in U.S. Pat. Nos. 6,607,751; 6,599,529; 6,569,463; 6,565,883; 6,482,440; 6,403,597; 6,319,919; 6,150,354; 6,080,736; 5,672,356; 5,472,704; 5,445,829; 5,312,817; and 5,296,483. Those skilled in the art will readily recognize other applicable sustained release formulations.

Parenteral routes may also be used, such as, inhalation of an recombinant microorganism formulation particularly for delivery to lungs or bronchial tissues, throat, or mucous membranes of the nose. Inhalable preparations include inhalable powders, propellant-containing metered dose aerosols, or propellant-free inhalable solutions. Inhalable preparations that can be used in delivering recombinant microorganisms are well-known in the art, for example, those inhalable preparations described in U.S. Pat. Nos. 7,867,987.

Recombinant microorganisms can also be formulated for transmucosal and transdermal administration. For transmucosal and transdermal administration, e.g., topical administration, recombinant microorganisms are formulated into a spray, gel, cream, foam, lotion, ointment, salve, powder, or suppository. Penetrants appropriate to the barrier to be permeated are used in the formulation. For example, the transmucosal and transdermal delivery agent can be, for example, DMSO, urea, 1-methyl-2-pyrrolidone, oleic acid, or a terpene (e.g., l-menthol, d-limonene, RS-(+/−)-beta-citronellol, geraniol). Further percutaneous penetration enhancers are described, for example, in Percutaneous Penetration Enhancers, Smith and Maibach, eds., 2nd edition, 2005, CRC Press. Exemplified transmucosal and transdermal delivery formulations that can be used in delivering recombinant microorganisms include those described in U.S. Pat. Nos. 6,589,549; 6,544,548; 6,517,864; 6,512,010; 6,465,006; 6,379,696; 6,312,717; and 6,310,177.

Recombinant microorganisms may be applied to the vagina in any conventional manner, including aerosols, foams, jellies, creams, suppositories, tablets, tampons, etc. Compositions suitable for application to the vagina are disclosed in U.S. Pat. Nos. 2,149,240; 2,330,846; 2,436,184; 2,467,884; 2,541,103; 2,623,839; 2,623,841; 3,062,715; 3,067,743; 3,108,043; 3,174,900; 3,244,589; 4,093,730; 4,187,286; 4,283,325; 4,321,277; 4,368,186; 4,371,518; 4,389,330; 4,415,585; and 4,551,148. The present invention may be carried out by applying recombinant microorganisms to the vagina in the form of such a composition. Suitable devices for applying recombinant microorganisms to the vagina are disclosed in U.S. Pat. Nos. 3,826,828; 4,108,309; 4,360,013; and 4,589,880.

Recombinant microorganisms may be applied to the anus in any conventional manner, including a foam, cream, jelly, etc., such as those described above with regard to vaginal application. In the case of anal application, it may be preferred to use an applicator that distributes the composition substantially evenly throughout the anus. For example, a suitable applicator is a tube 2.5 to 25 cm, preferably 5 to 10 cm, in length having holes distributed regularly along its length.

Also included in the invention are recombinant microorganism formulations for vaginal or anal administration with a solid carrier. These formulations may be presented as unit dose suppositories. Suitable carriers include cocoa butter and other materials well-known in the art. The suppositories may be conveniently formed by admixture of the recombinant microorganisms with the softened or melted carrier(s) followed by chilling and shaping in moulds.

In embodiments of the invention, the recombinant microorganism formulation is topically applied to the vagina or anus to prevent HIV infection as a result of vaginal or anal intercourse. Topical application is carried out prior to the beginning of intercourse, for example 0 to 60 minutes, 0 to 30 minutes, and 0 to 5 minutes.

In embodiments of the invention, recombinant microorganisms are released from an article when the article is placed on an appropriate body part or in an appropriate body cavity. For example, the invention includes IUDs, vaginal diaphragms, vaginal sponges, pessaries, or condoms that contain or are associated with (e.g., coated) an recombinant microorganisms.

In embodiments of the invention, an IUD contains or is associated with one or more recombinant microorganisms. Suitable IUDs are disclosed in U.S. Pat. Nos. 3,888,975 and 4,283,325. In embodiments of the invention, an intravaginal sponge contains or is associated with one or more recombinant microorganisms. In related embodiments, the intravaginal sponge releases the recombinant microorganisms in a time-controlled fashion. Intravaginal sponges are disclosed in U.S. Pat. Nos. 3,916,898 and 4,360,013. In embodiments of the invention, a vaginal dispenser contains or is associated with one or more recombinant microorganisms. Vaginal dispensers are disclosed in U.S. Pat. No. 4,961,931.

In embodiments, a condom contains or is associated with one or more recombinant microorganisms. In related embodiments, the recombinant microorganism is incorporated into the condom. In related embodiments, the condom is coated with a recombinant microorganism. In related embodiments, the recombinant microorganism is provided in a separate container, e.g., a package, and can be applied onto (e.g., outside and inside) the condom before the condom is used. In related embodiments, the condom is coated with a lubricant or penetration enhancing agent that comprises a recombinant microorganism. Lubricants and penetration enhancing agents are described in U.S. Pat. Nos. 4,537,776; 4,552,872; 4,557,934; 4,130,667, 3,989,816; 4,017,641; 4,954,487; 5,208,031; and 4,499,154.

Recombinant microorganism formulations suitable for topical administration in the mouth include lozenges comprising the recombinant microorganism in a flavored base, usually sucrose and acacia or tragacanth; pastilles comprising the recombinant microorganism in an inert base such as gelatin and glycerin, or sucrose and acacia; and mouthwashes comprising the recombinant microorganism in a suitable liquid carrier. In embodiments of the invention, recombinant microorganism is administered in the form of a mouthwash or gargle to prevent infection during dental procedures. The mouthwash or gargle is applied just prior to the beginning of the dental procedure and optionally periodically throughout the procedure.

The amount of the pharmaceutical composition administered will, of course, be dependent on the subject being treated, on the subject's weight, the severity of the disorder, e.g., stage of virus-mediated pathology, the manner of administration, and the judgment of the prescribing physician. Determination of an effective amount is well within the capability of those skilled in the art, especially in light of the detailed disclosure provided herein. Generally, an efficacious or effective amount of a recombinant microorganism is determined by first administering a low dose of the recombinant microorganism and then incrementally increasing the administered dose or dosages until a desired effect of reduced viral titer is observed in the treated subject, with minimal or acceptable toxic side effects. Applicable methods for determining an appropriate dose and dosing schedule for administration of a pharmaceutical composition of the present invention are described, for example, in Goodman and Gilman's The Pharmacological Basis of Therapeutics, Goodman et al., eds., 11th Edition, McGraw-Hill 2005, and Remington: The Science and Practice of Pharmacy, 20th and 21st Editions, Gennaro and University of the Sciences in Philadelphia, Eds., Lippencott Williams & Wilkins (2003 and 2005).

### Kits

The invention also provides for a pharmaceutical pack or kit for inhibiting viral infection. In embodiments, the kit comprises a recombinant microorganism as described herein. In embodiments, the kit comprises one or more containers filled with one or more of the ingredients of a recombinant microorganism formulation. Associated with such container(s) can be a notice in the form prescribed by a governmental agency regulating the manufacture, use, or sale of pharmaceuticals or biological products, which notice reflects approval by the agency of manufacture, use or sale of the kit and the components therein for human administration.

In embodiments, the kit comprises instructions for using the recombinant microorganism to inhibit viral infection using any of the methods described herein. In embodiments, the kit comprises instructions for using a recombinant microorganism in combination with at least one additional recombinant microorganism to inhibit viral infection using any of the methods described herein

The invention also provides that the recombinant microorganism formulation be packaged in a hermetically sealed container such as an ampoule or sachette indicating the quantity of composition. In embodiments, a recombinant microorganism composition is supplied as a liquid. In other embodiments, a recombinant microorganism composition is supplied as a dry sterilized lyophilized powder or water free concentrate in a hermetically sealed container and can be reconstituted, e.g., with water or saline, to the appropriate concentration for administration to a subject. In yet further embodiments, a recombinant microorganism composition is supplied as a gel, cream, foam, and the like.

### EXAMPLES

It should be appreciated that the invention should not be construed to be limited to the examples that are now described; rather, the invention should be construed to include any and all applications provided herein and all equivalent variations within the skill of the ordinary artisan.

### Example 1

### The Role of Cell-Associated Vs. Cell-Free Virus to Transmit HIV-1 Across an Epithelial Barrier

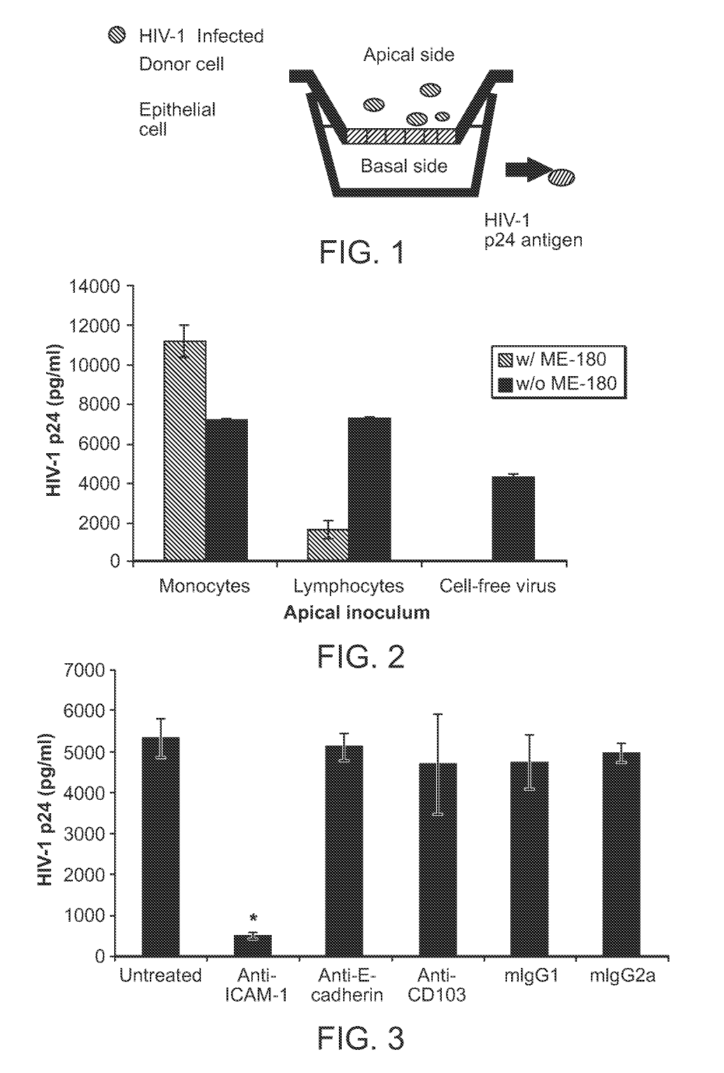

Initial studies were conducted using a transwell system (FIG. 1), in which free virus or cell-associated virus was placed in the upper of two chambers of a tissue culture system, and in which the two chambers are separated by a nylon mesh. A cervical epithelial cell line has been grown to confluence on the mesh as measured by electrical resistance across the chambers. This system permits study of the movement of virus across epithelium to the interepithelial or submucosal dendritic cells in which initial infection of host cells is established (Hu et al, J Virol 74:6087-6095 (2000); and Spira et al., J Exp Med 183:215-225 (1996)). Infected cells or cell free virus are placed in the apical chamber with or without reagents that might inhibit transmission. After 24 hours, a sample from the lower chamber is examined for the presence of HIV-1 p24 antigen.

One of the advantages of this system compared to the explant-based transwell systems is the maintenance of epithelial integrity over a period of several days, with transmission of horse-radish peroxidase across the epithelium at less than 1% of that added to the apical chamber, compared to the 7-8% transmission rate observed in the explant systems. While the explant system may be useful for identifying targets of infection in the sub-epithelial mucosa and for evaluating the ability of candidate microbicides to block that infection (Cummins et al., Antimicrob Agents Chemother 51:1770-1779 (2007)), it is probably suboptimal for studying movement across epithelium.

Using this system, the relative ability of cell-free vs. cell associated virus to cross this epithelial barrier was first examined (FIG. 2). Clearly, without the epithelial cells present, both cell-associated and cell-free virus move freely across the nylon mesh separating the two chambers. For this study ME-180 cervical epithelial cells were grown to confluence on the mesh. Once the epithelial barrier was confluent, cell-free virus (cfv) failed to move across. Subsequent studies revealed that cfv could be detected in the basilar chamber when human, rather than fetal calf, serum was used in the media (see also Kage et al., J Virol 72:4231-4236 (1998), although transmission of cfv was never as efficient as cell-, and particularly macrophage-, associated virus.

### Example 2

### Ability of Antibodies Targeting Molecules Involved in Cell Translocation Across Epithelia to Block HIV-1 Transmission

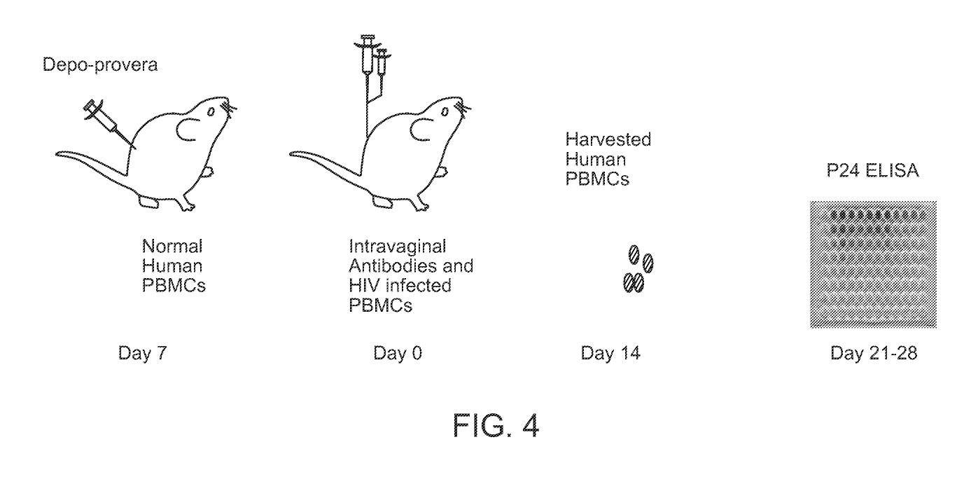

Because of the potential importance of cell-associated transmission of virus across epithelium, the ability of antibodies targeting ligands that were potentially involved in that process to block transmission in HIV-1 infected cells was evaluated using the transwell assay. All of the antibodies were derived from mouse hybridomas, purified on Protein G columns, and protein concentrations were determined using the Bio-Rad protein assay (Hercules, Calif.). As seen in FIG. 3, only antibody to ICAM-1 significantly reduced transmission in this system.

### Example 3

### Ability of Anti-ICAM-1 to Block Vaginal Transmission of Cell-Associated HIV-1 in a Mouse Model

Because mice cannot be infected with HIV-1, the inventors developed a vaginal transmission model in which CB.17 scid/scid immunodeficient mice receive intraperitoneal transplantation of human peripheral blood mononuclear cells and then are challenged by the vaginal route with HIV-1 infected PBMC or macrophages (HuPBL-SCID mouse model, FIG. 4). In order to enable transmission in this system, the mice must first be pre-treated with progesterone, which converts the stratified squamous epithelium of the vagina into the single-layered columnar epithelium typical of the endocervix, thought to be a “hot zone” of HIV-1 transmission (Anderson et al., N. Engl. J. Med 309:984-985 (1983)). At the time of progesterone treatment 5×10⁷ normal uninfected, unstimulated PBMC are placed into the peritoneal cavity. One week later, mice receive 1×10⁶ PBMC or macrophages by atraumatic intravaginal inoculation 15 minutes after administration of experimental or control antibodies at the indicated concentrations. The cells and the antibody are each administered in 10 μl of PBS. Two weeks later the mice are euthanized and peritoneal cells are harvested, which are then co-cultured with activated PBMC to determine if the intravaginally administered HIV-1 was able to pass through the epithelium and infect the previously transplanted cells in the peritoneal cavity. Studies have indicated that intravaginally inoculated PBMC can be recovered from paraaortic lymph nodes within 15 minutes of being inoculated intravaginally. The results of a representative experiment are shown in Table 1.

TABLE 1
Ability of antibodies to adhesion molecules to block HIV-1
transmission in the Hu-PBL-SCID mouse model

| | HIV-positive |
|---|---|
| Treatment | mice/total |
| Anti-mouse-ICAM-1 | 0/8 (0%), p <0.01 |
| (0.01 ml @ 20 ug/ml) | |
| Anti-human-CD18 | 0/8 (0%), p <0.01 |
| (0.01 ml @ 20 ug/ml) | |
| Isotype control Ab | 6/7 (86%), * |
| (0.01 ml @ 20 ug/ml) | |

Given that anti-ICAM-1 blocked transmission, it was reasonable to ask if anti-CD18, the beta chain of the LFA-1 heterodimer (CD11a/CD18) that is the counter-receptor for ICAM-1, would also be effective, and, as also shown in Table 1, it was. See also U.S. Patent Application Publication No. 2009/0317404 A1, which is hereby incorporated by reference in its entirety. It should be noted that, in studies not shown, anti-human ICAM-1 did not work in mouse studies, indicating that the ICAM-1 being recognized resides on the mouse epithelial cells, while the LFA-1 that is effectively targeted in this system is of human origin.
Human LFA-1 can engage murine ICAM-1, although the converse is not true (Johnston et al., J Immunol 145:1181-1187 (1990)).

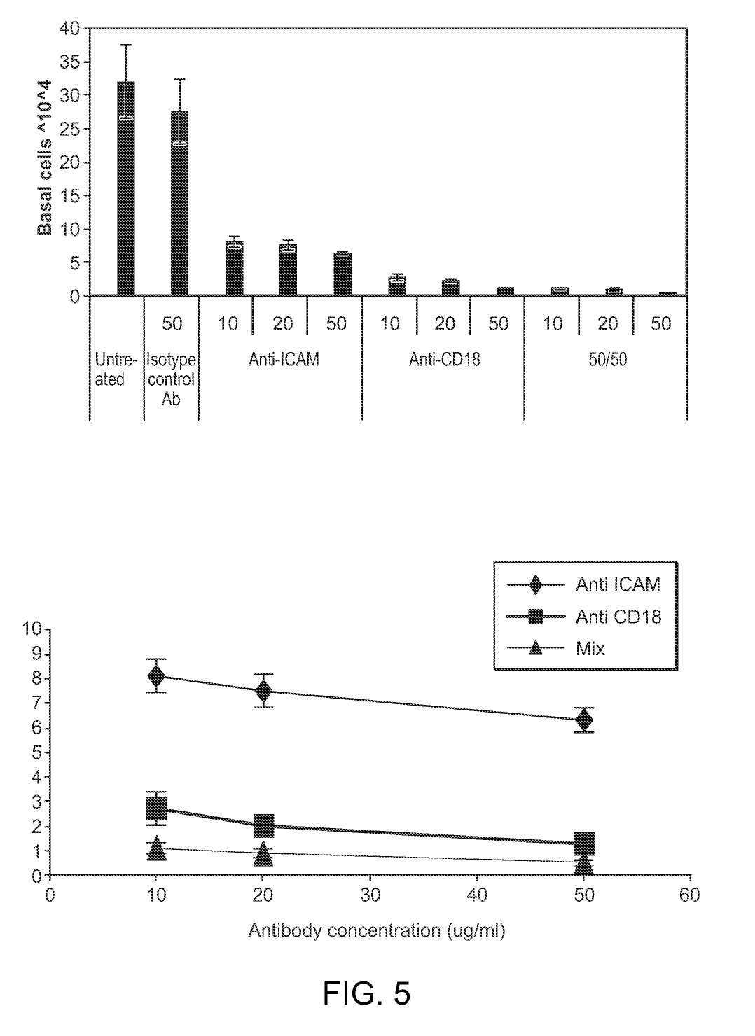

Since both anti-CD18 and anti-ICAM-1 were effective in blocking transmission, the antibodies were then assessed to determine if the combination of the two antibodies might exert additive blocking activity. This additive activity is evident in FIG. 5. When the combined antibodies are used, each of the antibodies is present in half of the concentration cited, e.g., 10 ug/ml of antibody represent 5 ug/ml of each of the individual antibodies. This finding is important because it indicates that a combination of antibodies may be efficacious at antibody concentrations that have been able to achieve in culture.

### Example 4

### Lack of Toxicity Associated with Sustained Anti-Mouse ICAM-1 Intravaginal Administration

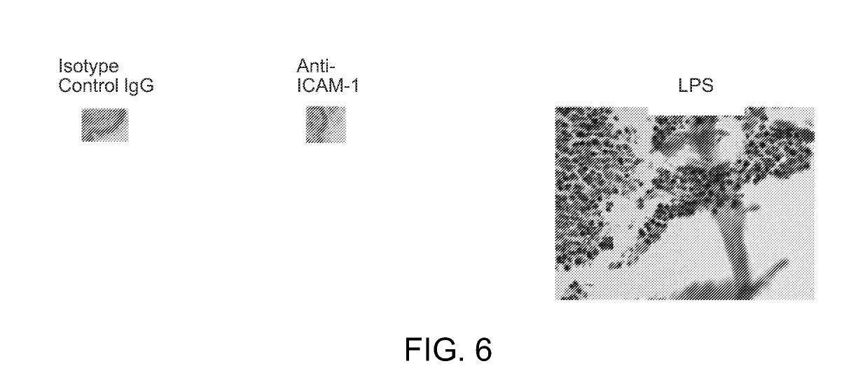

To determine if persistent administration of anti-ICAM-1 could elicit an inflammatory response, three mice were administered this antibody intravaginally twice daily at a concentration of 100 μg/administration for 14 consecutive days. Control mice received an irrelevant isotype-control antibody at the same concentration or, as a positive control, Salmonella typhi lipopolysaccharide (LPS) at a concentration of 1 μg/ml. While histopathological examination of the vagina of the mice revealed a profound inflammatory response to the LPS, none was observed in the mice treated with anti-ICAM-1. Representative slides are show in FIG. 6.

### Example 5

### Blocking of Cell-Associated Transmission by ICAM-1 does not Require the Fc Region of the Ig Molecule

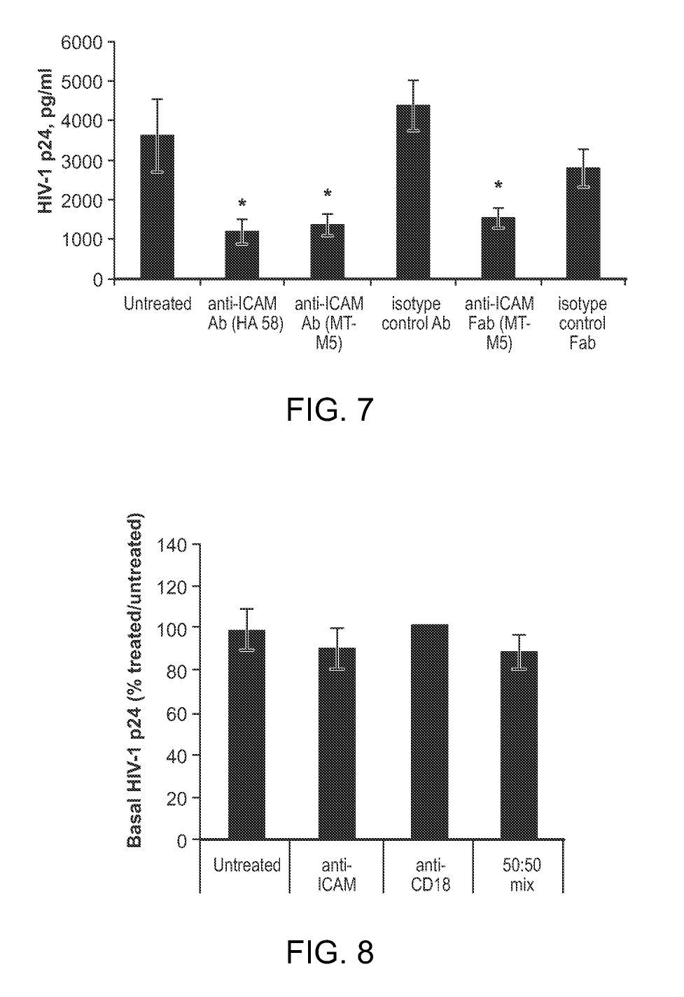

In order for lactobacillus scFv to be effective, the transmission-blocking activity of the monoclonal antibody should not be dependent on the presence of the Fc region of the molecule. Fab, prepared from the monoclonal anti-ICAM-1 antibody using the ficin-based ImmunoPure Fab/F(ab′)₂ digestion kit (Pierce), was therefore tested to block transmission in the transwell assay. As can be seen in FIG. 7, the Fab retained transmission blocking capability. These studies laid the groundwork for generation of scFv to be expressed in lactobacilli.

### Example 6

### Anti-CD18 Antibody Blocks Infection by Cell-Free Virus

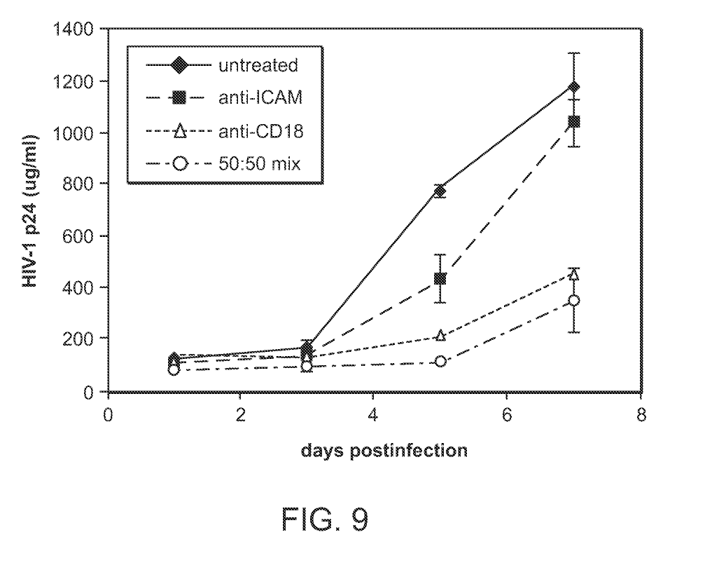

As indicated above, transmission of cell-free HIV-1 (cfv) is observed in the transwell assay if human serum, rather than FBS, is used in the tissue cultures. Under these conditions the ability of anti-human ICAM-1 and anti-human CD18 to block movement of free virus across the epithelial barrier was evaluated. As shown in FIG. 8, neither of these antibodies could block the movement of cell free virus across the epithelium. It was then determined whether virus exposed to these antibodies in the upper chamber of a transwell could pass through the epithelium and establish infection of susceptible cells in the lower chamber. As shown in FIG. 9, both of these antibodies and the combination of them, but particularly anti-CD18, were able to reduce the establishment of infection in the cells beneath the epithelial layer. This was true despite the exposure of the cultures to a very large inoculum of virus (10³ TCID₅₀). Thus, antibodies to adhesion molecules and/or their integrin counter-receptors are able to significantly reduce productive HIV-1 infection resulting from exposure to both cell-associated and cell-free virus. It is worth emphasizing that, although 100% protection is not observed in the transwell assay, the levels of transmission reduction observed are typically associated with almost complete protection in the Hu-PBL-SCID system.

The ability of CD18 antibodies is significant as an effective method for preventing HIV transmission will likely have to inhibit both cell-associated and cell-free virus. Although studies in macaques, in which cell-associated virus has been poorly transmitted while cell-free virus is much more readily transmitted (Sodora et al., AIDS Res Hum Retroviruses 14 Suppl 1:S119-123 (1998); Miller et al., J Virol 68:6391-6400 (1994); Miller et al., J Med Primatol 21:64-68 (1992); Miller et al., J Virol 63:4277-4284 (1989); and Hu et al., J Virol 74:6087-6095 (2000)), have generated a focus on cell-free virus, that may not represent the true transmission setting. Almost all of these studies are not conducted in the presence of seminal plasma, which, with its alkaline pH would neutralize the normally acidic environment of the macaque vagina. In even mildly acidic environments, cells lose their ability to translocate (Rotstein et al., J Surg Res 45:298-303 (1988)). Studies in chimpanzees have demonstrated that close approximation of HIV-1 infected cells to the cervix does results in transmission of infection (Girard et al., AIDS Res Hum Retroviruses 14:1357-1367 (1998)). In addition, modeling vaginal retrovirus transmission using cats challenged with feline immunodeficiency virus, Bishop et al. (Vet. Microbiol. 51:217-227 (1996)) found that cell-associated virus was transmitted with greater frequency than cell-free virus. As such, it is clear that a conservative approach to preventing HIV-1 transmission should target both cell-associated and cell-free virus.

### Example 7

### A Mouse Model for Evaluating Cell-Free HIV-1 Transmission In Vivo

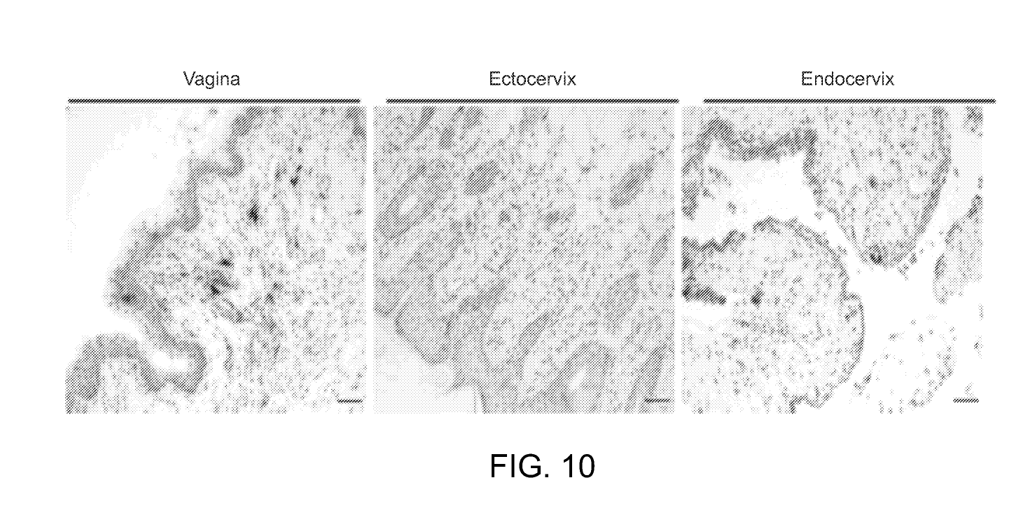

As indicated, in the Hu-PBL-SCID mouse model described above, only transmission of cell-associated virus is observed. This model allows examination of such transmission isolated from cell-free transmission, since free virus is not transmitted in this model system. However, because it is likely that both cell-associated and cell-free virus play a role in the transmission process, it would be important to demonstrate the in vivo efficacy of these antibodies in blocking cell-free virus transmission. The laboratory of Dr. J. Victor Garcia, has developed a mouse model for examining cell-free HIV-1 transmission using non-obese diabetic SCID mice transplanted with human fetal thymus, fetal liver, and bone-marrow derived CD34+ stem cells (Melkus et al., Nat Med 12:1316-1322 (2006); Wege et al., A. K., Curr Top Microbiol Immunol 324:149-165 (2008); and Denton et al., PLoS Med 5:e16 (2008)). They have termed this mouse the BLT mouse (bone marrow, liver, thymus) to distinguish it from other SCID-hu mouse models that do not involve bone marrow transplantation. Numerous studies have indicated the importance of dendritic cells for cell-free virus transmission (Hu et al., J Virol 74:6087-6095 (2000); Girard et al., AIDS Res Hum Retroviruses 14:1357-1367 (1998); Spira et al., J Exp Med 183:215-225 (1996); Hu et al., Lab Invest 78:435-451 (1998); and Miller et al., Am J Pathol 141:655-660 (1992)) and the presence of these cells in the lamina propria of the genitourinary tract of transplanted mice likely accounts for the ability of this model system to support cell-free transmission. FIG. 10 demonstrates the presence of CD11c+ dendritic cells in reconstituted mice. The Garcia laboratory has reported on their ability to establish cell-free transvaginal HIV-1 transmission in this system and on the ability of prophylactic antiretrovirals to block this transmission (Denton et al.).

### Example 8

### Generation of scFv in Lactobacilli

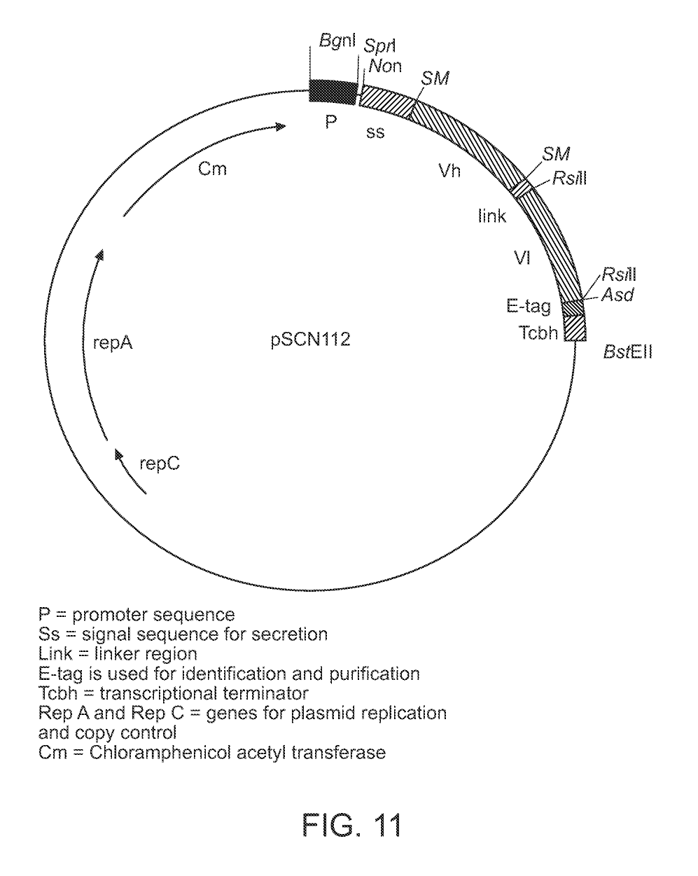

Initial generation of scFv was undertaken using a plasmid-based expression system to determine if the secreted scFv retained antigen binding activity and were active in the transwell assay. Following RNA isolation from the MT-M5 (anti-human ICAM-1 producing) hybridoma and cDNA preparation, the variable H chain and variable L chain regions from the anti-ICAM-1 hybridoma MT-M5 were amplified via PCR and cloned into the vector pSCN112 as illustrated in FIG. 11. The resulting plasmid pSCN112hIcam-1 was then electroporated into L. casei 393 as follows: L. casei 393 was grown at 37° C. overnight in Mann-Rogosa-Sharpe medium (Difco; BD Biosciences). The overnight culture was diluted 50-fold in fresh MRS and incubated at 37° C. for another five hours (OD₆₀₀=0.8). Cells were collected through centrifugation, washed three times with electroporation wash buffer (5 mM NaH₂PO₄, pH 7.4, 1 mM MgCl₂), and washed once with electroporation buffer (0.3 M sucrose, 5 mM NaH₂PO₄ (pH 7.4), and 1 mM MgCl₂). Cells were resuspended in 1/100 volume electroporation buffer. Transformation was conducted via electroporation of 50 μl of cells with 200 ng of plasmid DNA, and transformants were selected on Mann-Rogosa-Sharpe plates with chloramphenicol at 10 μg/ml. Transformant lactobacilli were tested for expression of scFv using both Western blot analysis and detection of E-tag, a FLAG sequence that was translationally fused to the scFv for detection and purification purposes. Transformed lactobacilli were grown in Mann-Rogosa-Sharpe medium at 37° C., and secreted scFvs were purified using the E-tag HiTrap purification system (GE Healthcare).

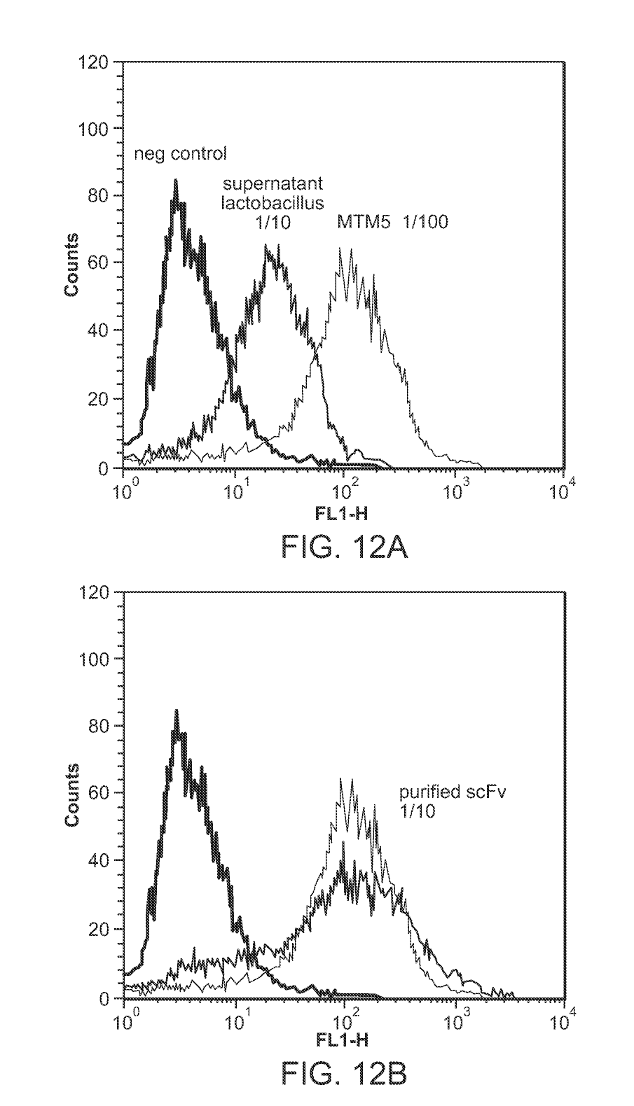

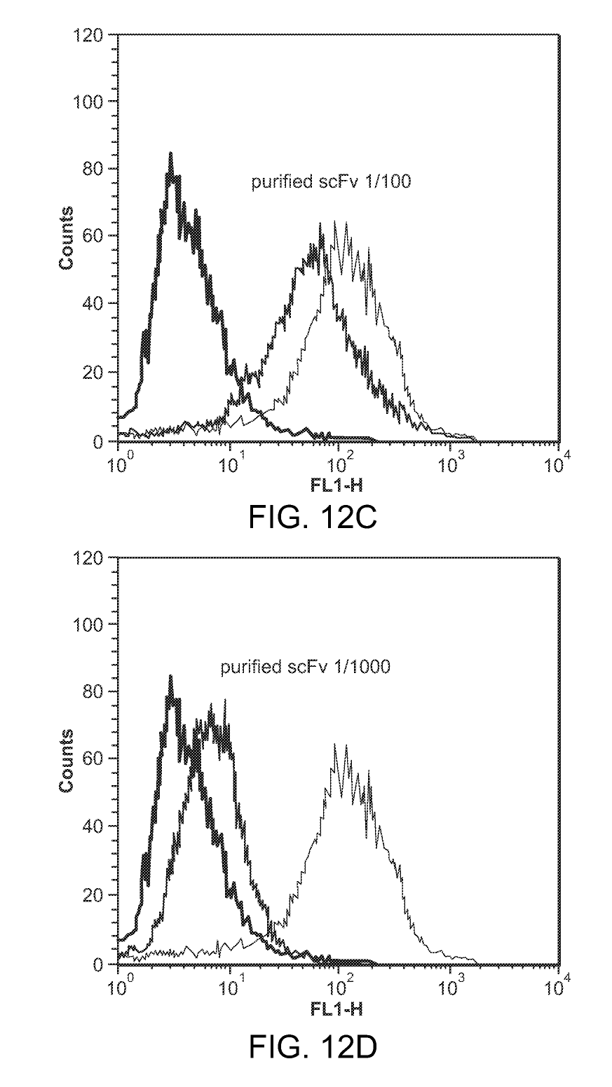

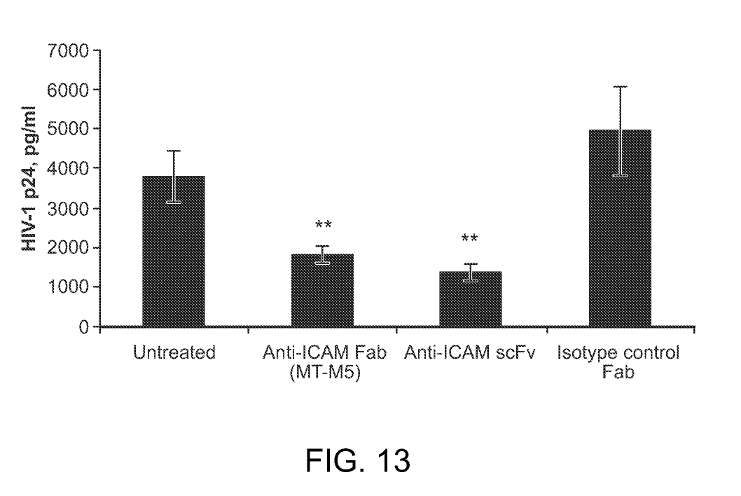

The binding capability of the scFv diluted directly from centrifuged and filtered broth is demonstrated in the flow cytometric analysis shown in FIGS. 12A-12D, in which binding to CHO cells expressing ICAM-1 is shown for the original hybridoma antibody, a 1/10 dilution of the filtered broth, and various dilutions of a 20 ug/ml stock of purified scFv prepared from lactobacillus culture broth. The scFv is clearly functional in this flow cytometric analysis and maintains functionality through a 1/100 dilution. The data in FIG. 13 demonstrate that these scFv are functionally equivalent in the transwell assay to the Fab prepared from the original monoclonal antibody. Because this antibody targeted human ICAM-1, it could not be evaluated in the Hu-PBL-SCID mouse model in which antibodies directed at mouse ICAM-1 are required. Efforts to generate secreted scFv against mouse ICAM-1 were unsuccessful for reasons that are unclear. While scFv to human CD18 were obtained using the plasmid expression system, the concentration of antibody was below 1 ug/ml in broth solution, suggesting that concentrations that would be expected to be effective cannot be achieved in situ, based on the results of our synergistic/additive assays with antibody to ICAM-1 and CD18. It is this unpredicatability of the scFv expression system that prompted the inventors to undertake generating the lactobacillus-based VHH expression system.

### Example 9

### Generation and Expression of Anti-Rotavirus VHH by Lactobacilli and their Use for In Situ Protection in a Rodent Model of Rotavirus-Induced Gastroenteritis

The potential therapeutic benefit of in situ production of pathogen-targeting VHH was previously demonstrated in a mouse model of rotavirus diarrhea (Pant et al., J Infect Dis 194:1580-1588 (2006)) Llama VHH produced by lactobacilli was shown to be effective in controlling rotavirus diarrhea. The VHH were prepared as described (van der Vaart et al., Vaccine 24:4130-4137 (2006)). Briefly, a llama was immunized multiple times with rotavirus in an oil emulsion. The immune response was followed by titration of serum samples in ELISA with rotavirus coated at a titer of 4×10⁶ pfu/ml. On day 153 after immunization, peripheral blood lymphocytes were collected, RNA was extracted, cDNA generated and VHH genes were amplified with primers Lam-16 (GAGGTBCARCTGCAGGASAGYGG), Lam-17 (GAGGTBCARCTGCAGGASTCYGG), Lam-07 (priming to the short hinge region) and Lam-08 (long hinge specific). The amplified products were digested with PstI and NotI (New England Biolabs) and cloned in phagemid vector pUR5071, which is based on pHEN1 (Hoogenboom et al., Nucleic Acids Res 19:4133-4137 (1991)), and contains a hexahistidine tail for Immobilized Affinity Chromatography and a c-myc derived tag for detection. Ligation and transformation were performed as described (de Haard et al., J Biol Chem 274:18218-18230 (1999); and Hoogenboom et al., Immunotechnology 4:1-20 (1998)). The rescue with helper phage VCS-M13 and PEG precipitation was performed as described in Marks et al., J Mol Biol 222:581-597 (1991). Selections were performed via the biopanning method (Marks et al.) by coating of rotavirus strain RRV (2.5×10⁷ pfu/ml at round 1; 5×10⁴ pfu/ml at round 2; 500 pfu/ml at round 3). Immunotubes were coated overnight at 4° C. with either a 1:1000 dilution of anti-rotavirus rabbit sera or anti-rotavirus guinea pig sera in carbonate buffer (16% (v/v) 0.2 M NaHCO3+9% (v/v) 0.2 M Na2CO3). Viral particles were captured via polyclonal anti-rotavirus sera. Subsequently, the standard selection procedure was followed (Marks et al.). Soluble VHH was produced by individual E. coli TG1 clones as described in Marks et al. Culture supernatants were tested in ELISA. After incubation of the VHH containing supernatants, VHH's were detected with a mouse anti-myc monoclonal antibody.

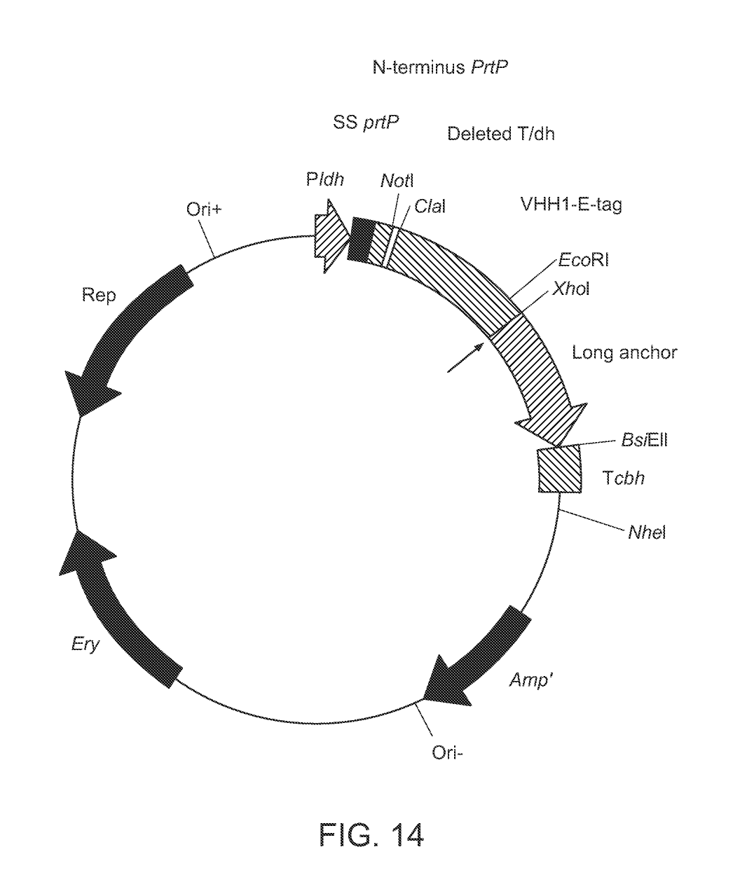

For the generation of the surface-expressed antibody fragment on lactobacilli, the DNA fragment coding for VHH1-E-tag gene (E-tagged for detection and purification) was fused to an anchor sequence (the last 244 aa of the proteinase P protein of Lactobacillus casei) and into the Lactobacillus expression vector pLP501 (FIG. 14) cut with restriction enzymes ClaI and XhoI. To generate the secreted VHH1 antibody fragment, a stop codon (TAA) was inserted by polymerase chain reaction (PCR) amplification after the E-tag, and the product was introduced into pLP501. Transformation of L. paracasei (previously named L. casei ATCC 393 pLZ15) was performed as described elsewhere (Kruger et al., Nat Biotechnol 20:702-706 (2002)). In both constructs, the expression is under the control of the constitutive ldh promoter of L. casei (Pouwels et al., J Biotechnol 44:183-192 (1996)). Lactobacillus expressing an irrelevant VHH-secreted fragment (directed against a Lactococcus phage protein) and an irrelevant VHH-anchored fragment (directed against the SA I/II adhesin of S. mutans) were constructed in a similar way and used as controls.

Mice were then fed 10⁸ of the different lactobacillus constructs from days −1 to +3. On Day 0, mice were challenged with 20 diarrheal doses₅₀ of rotavirus. The data in Table 2 indicate that reduction in disease severity and duration was provided by lactobacilli that expressed a membrane anchored version of the anti-rotavirus VHH.

TABLE 2
Duration and severity of diarrhea in the different treatment groups

| | Mice, | Duration,^{b} | Severity scores,^{c} |
|---|---|---|---|
| Treatment group | no.^{a} | mean ± SE, days | mean ± SE [symbol] |
| Untreated | 30 | 2.13 ± 0.10 | 3.73 ± 0.16 |
| Lactobacillus paracasei | 17 | 1.94 ± 0.20 | 2.88 ± 0.35 |
| VHH1-secreted | 10 | 1.91 ± 0.16 | 2.73 ± 0.23 |
| Irrelevant VHH-anchored^{d} | 17 | 2.12 ± 0.17 | 3.35 ± 0.28 |
| VHH1-anchored | 27 | 1.22 ± 0.16^{e} | 1.67 ± 0.25^{f} |
| Lyophilized VHH1-anchored | 7 | 1.28 ± 0.28 | 1.86 ± 0.40^{g} |

NOTE: VHH, variable domain of Ilama heavy-chain.
^{a}The no. of mice were pooled from all experiments.
^{b}Duration was defined as the sum total of days with diarrhea.
^{c}Watery diarrhea was given a score of 2, loose stool a score of 1, and no stool or normal stool a score of 0. Severity was defined as the sum of diarrhea scores for each pup during the course of the experiment (severity = Σ diarrhea score [day 1 + day 2 + day 3 + day 4]).
^{d}Directed against the SA VII adhesin of Streptococcus mutans.
^{e}Compared with untreated, P <.01; compared with irrelevant, P <.05 (Kruskal-Wallis and Dunn tests for both).
^{f}Compared with untreated, P <.001; compared with irrelevant, P <.01 (Kruskal-Wallis and Dunn tests for both).
^{g}Compared with untreated, P <.05 (Kruskal-Wallis and Dunn tests). [symbol]
indicates data missing or illegible when filed

The number of mice that developed any diarrhea was also significantly lower in the group to which the lactobacilli carrying membrane-anchored anti-rotavirus VHH was administered (40% vs. 100%). Transformed lactobacilli were recovered from the intestinal tract of the mice 48, but not 96, hours after administration of the final dose, independent of transformation status (Pant et al.).

### Example 10

### Alpaca Immunization Studies

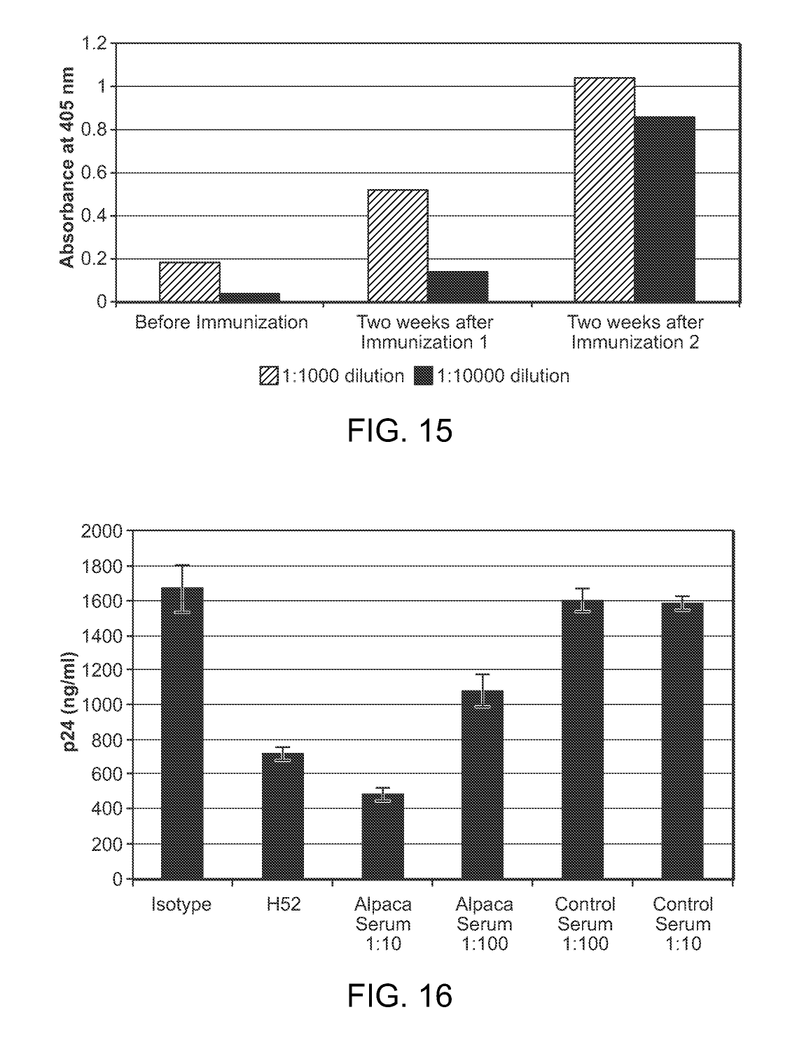

Because alpacas are smaller and easier to manage than llamas, VHH from alpacas were generated (see Maass et al., J Immunol Methods 324:13-25 (2007)). For these studies, exons 5-8 of CD18, constituting those portions of the ectodomain of this molecule that are engaged by ICAM-1, were expressed. Using primers 5′CD18 Ex4-5 BglII GATCAGATCTCTGCGACC-AGGCCAGGCAGC and 3′CD18 Ex8-9 SalI CTAGTCGACCTCGGTGAGTTTCTCGTAGG for subcloning into the vector pGEX-4T-1 and CD18 5-8 5′BamHI CCAGGATCCCTGCGACCAGGCCAG-GCAGC CD18 5-8 3′HindIII CGGAAGCTTTTACTCGGTGAGTTTCTCGTAGGTC for subcloning into the vector pQE30, CD18 constructs were expressed in E. coli D5α cells. CD18 were purified in quantities sufficient to immunize a single alpaca on four occasions at two week intervals (400 μg/immunization in complete followed by incomplete Freund's adjuvant). Serum obtained two weeks after the second immunization was evaluated by ELISA for anti-CD18 antibodies (FIG. 15). The activity of the alpaca sera was also examined in the transwell assay. As seen in FIG. 16, a 1:10 dilution of the alpaca serum inhibited transmission more effectively than did the anti-CD18 Mab used at a concentration of 20 ug/ml. As the results indicate that a strong protective humoral response is elicited by this immunization protocol, lymphocytes were harvested from peripheral blood and VHH cDNA were generated. VHH were made from the VHH cDNA and screened with the phage screening assay, as described in detail below.

### Example 11

### VHH Targeting ICAM-1 and CD18

VHH directed to ICAM-1 and CD18 are obtained as follows. See also Maaas et al.

### 1. Preparation of Alpaca Lymphocytes

Peripheral blood lymphocytes (PBL) are partially purified by separation over Ficoll-Paque (Invitrogen) using standard procedures and stored in RNAlater (Qiagen). Serum is separated by centrifugation and stored at −20° C. until testing.

### 2. Alpaca Immunization Evaluation of Humoral Immune Response

Adult female alpaca are given four immunizations at two week intervals, each including six 0.2 ml intra-dermal and subcutaneous injections of the immunogen (e.g., ICAM-1, CD18, and fragments thereof) in the pre-scapular region. The innoculum contains about the same amount of immunogen as for the GST-CD18(Ex5-8) antigen described above (about 400 μg of GST-CD18(Ex5-8)) for each immunization prepared with same volume of Freund's adjuvant. Serum samples are obtained one week after the immunization, and tested for antibody response by ELISA and transmit assay. Using this immunization protocol, alpaca antibody bound to GST-CD18(Ex5-8) was detected in ELISA using HRP labelled anti-llama IgG (H+L) (Bethyl), which is cross-reactive with alpaca antibody.

3. Alpaca cDNA Preparation and VHH Cloning

After removing RNAlater from PBL or lymph node tissue, total RNA is isolated from PBL using TRIZol Reagent (Invitrogen) according to the manufacturer's protocol. Then RNA is column-purified using an RNeasy Mini Kit (Qiagen) and stored at −80° C. First-strand cDNA synthesis is performed using SuperScript II RNAse H⁻ reverse transcriptase (Invitrogen) and a combination of poly(A) oligo(dT)12-18 and pd(N)₆ primers.

VHH coding DNA for the phage display library is amplified from alpaca cDNA using primer pairs containing a mixture of two CH2 reverse primers:

AlpVHH-R1 (GATCACTAGTGGGGTCTTCGCTGTGGTGCG)
and
AlpVHH-R2 (GATCACTAGTTTGTGGTTTTGGTGTCTTGGG)
with
AlpVh-F1 (GATCGCCGGCCAGKTGCAGCTCGTGGAGTCNGGNGG)

as the forward primer. After being purified via agarose electrophoresis using QIAquick Gel Extraction kit (Qiagen), amplified VHH DNA is digested with appropriate restriction enzymes and ligated into similarly digested phage display vector pCANTAB 5E (GE Healthcare). The ligated DNA is transformed by electroporation into high efficiency electroporation-competent TG1 cells (Stratagene). Transformants are scraped off the plates and recombinant phage are produced according to standard methods.

A quality check is made for the library by selecting 40 random clones for PCR amplification using primers flanking the VHH cloning site. Each PCR product is analyzed for size by agarose gel electrophoresis and digested with BstN1 to assess the “fingerprint” fragment patterns (Tomlinson et al., J Mol Biol 227:776-798 (1992)).

### 4. Screening and Selection of Phage Antibodies

Selection is carried out by panning of VHH-displayed phage libraries for phage that bind to immunotubes (Nunc) coated with the immunogen (e.g., 5 μg/ml soluble (His)₆-CD18(Ex5-8) was used in Example 10). The tubes are then washed three times with PBS, and blocked with 4% non-fat dried milk in PBS (MPBS) at 37° C. for 2 hours. A 4 mL suspension of 5.0×10¹¹ CFU phage in MPBS is incubated in an immunotube at room temperature for 30 minutes with continuous rotation, and then for a further 90 minutes without rotation. The tubes are washed 20 times with PBS containing 0.1% Tween 20 (PBST) followed by 20 times with PBS. Bound phage will be eluted by continuous rotation with 1 mL of 100 mM triethanolamine (Sigma) for 10 minutes, then, recovered and neutralized with 0.2 ml of 1 M Tris-HCl, pH 4.5. A 0.75 mL aliquot of the eluted phage is used to infect a 10 mL culture of log-phase E. coli TG1 cells. A small aliquot of the infected bacteria is used in serial dilutions to titrate the number of phage eluted while the remainder is processed to amplify the phagemid for further selection or analysis. The binding of selected VHHs encoded by phagemid clones to the immunogen (e.g., (His)₆-CD18(Ex5-8)) is tested by phage ELISA using anti-M13 antibody (GE Healthcare) for detection. Positive clones are then “fingerprinted” by analysis of their BstN1 digestion patterns.

### 5. Production and Validation of Soluble Single Domain Antibodies

To validate the production of the VHH, the VHH coding DNA is subcloned into the expression vector pQE30 (Qiagen), or any suitable expression vector well-known in the art. Transformed E. coli M15 (Qiagen) containing the VHH expression plasmid is grown to an optical density of 0.5 at 600 nm and protein expression is induced overnight in 1 mM IPTG at 30° C. Soluble protein is purified from sonicated cells and the recombinant VHH is purified as recommended by the manufacturer, e.g., by nickel affinity using Ni-NTA (Qiagen) if the immunogen has a histidine tag. Protein eluted, e.g., in 0.2 M imidazole, is dialyzed against PBS. Purity of the recombinant VHH is assessed by Coomassie Blue staining of SDS-PAGE and protein concentration determined by BCA (Pierce). Western blot and ELISA detection of recombinant VHH will be performed using an HRP antibody (e.g., HRP anti-His-tag antibody). If production of the immunogen-specific VHH is confirmed, the plasmid is introduced into the appropriate bacterial expression system (e.g., lactobacillus, including the species L. rhamnosus GR-1 and L. reuteri RC-14). Examples of suitable expression systems include the following:

a. Chromosomal Integration-Based Expression System

Vector systems based on the site-specific integration apparatus of temperate bacteriophage A2 of Lactobacillus have already been constructed in order to generate food grade modified Lactobacillus (Martin et al., Appl Environ Microbiol (2000)). The pEM76-based delivery system vector employed the phage A2 integrase gene (A2-int) which catalyzes the insertion of vector DNA containing the A2-attP site into an attB site present in the genome of all lactic acid bacteria. The pEM76-based delivery system does not replicate in Lactobacillus. In addition to the int gene and attP site, it contains an E. coli replication origin, the β-lactamase and erythromycin resistance genes flanked by two directly oriented six sites as well as a multicloning site where heterologous DNA (expression cassette) may be cloned. Following transformation, the whole plasmid integrates into the chromosome and a depuration system has been developed to eliminate the unwanted DNA. The recombinant strains are transformed again with a shuttle E. coli-lactic acid bacteria vector containing a β-recombinase gene. The β-recombinase catalyzes the deletion of the antibiotic resistance gene and the origin of replication contained between the two six site leaving in the chromosome only the int gene, one six site and the cloned DNA. The plasmid is then cured to render the strain plasmid free. The system can be applied to a range of Lactobacillus species and the whole procedure is simple. This method eliminates the need to know the genome sequence since the DNA always integrate at the attB site. The temperate phage being 20 kb probably long sequence of DNA can be integrated, allowing the integration of two or more expression cassettes encoding antibodies of different specificities. This system has previously been used to integrate the fusion between the apf gene and the gene encoding the scFv directed against the SAI/II adhesion of S. mutans. Similarly, the APF expression cassettes containing the VHH antibody gene can be cloned into the pEM76-based delivery system to mediate chromosomal integration of the expression cassette into the attB site of the model strain L. casei 393 and subsequently into the attB site of selected Lactobacillus strains that can colonize the vaginal tract

b. Plasmid Based Expression System

An alternative system for plasmid-based expression employs the aggregation promoting factor (APF) of Lactobacillus as the fusion partner, which mediates expression and export of the antibody fragments. The APF is a cell surface protein constitutively expressed by Lactobacillus. It is non-covalently attached to the surface of Lactobacillus as well as secreted at a high rate in the medium (Marcotte et al., J Appl Microbiol 97:749-756 (2004)) (102).

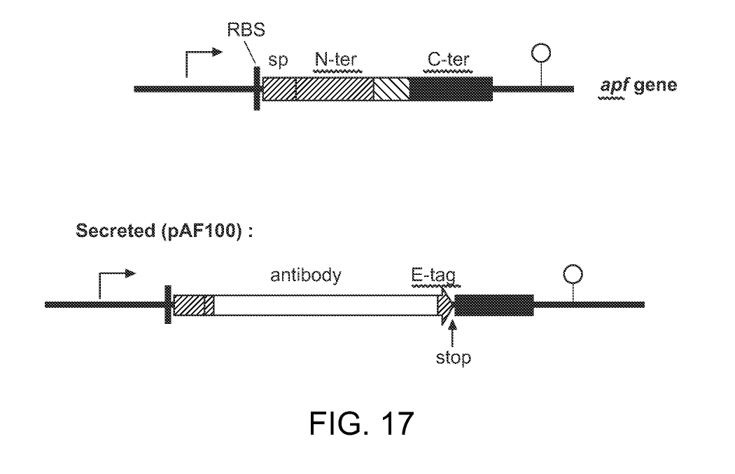

An expression variant of this gene has been constructed to generate Lactobacilli expressing VHH fragments secreted in the medium (pAF100) (FIG. 17) by insertion of a stop codon prior to the C terminus of the gene. The gene is inserted into the wide-host-range shuttle vector for lactic acid bacteria and E. coli pIAV7 (Perez-Arellano et al., Plasmid 46:106-116 (2001)). This plasmid has been shown to be structurally and segregationally stable through at least 120 generations in lactobacilli, and is able to maintain a high copy number (˜160) in L. casei (Perez-Arellano et al).

In order to prevent the spread of the genetically modified lactobacilli in the environment, the ThyA gene will be disrupted, as described in Steidler et al., Nat Biotechnol 21:785-789 (2003). The knockout of the thyA gene promotes the death of the strain in the absence of thymine or thymidine, ensuring biological containment of the modified strain within the vagina. Primers will be designed to amplify regions at the N-terminal and C-terminal region of the thyA gene. These regions will be cloned in the temperature sensitive plasmid pG+host9, which contains an erythromycin resistance gene. Following transformation of the selected Lactobacillus strain with the plasmid containing these thyA regions, the temperature will be raised to the non-permissive temperature for plasmid replication, enforcing integration of the plasmid in the thyA gene. The temperature will be returned to the permissive temperature to allow the plasmid to replicate and recombine out of the chromosome. A thy-, ery-phenotype will be selected and thyA knockout colonies will be determined via PCR and DNA sequence analysis. The viability of the strains in the presence and absence of thymidine will be evaluated. It should be noted that a similarly engineered Lactococcus strain expressing the immunomodulating cytokine IL-10 was used in the previously cited clinical trial (Braat et al., Clin Gastroenterol Hepatol 4:754-759 (2006)).

c. S. gordonii Expression Plasmids for Use in Mouse Models

Streptococcus gordonii expression plasmids are generated, and S. gordonii transformed with the plasmid express secreted VHH using PLEX. This plasmid was developed from the commercially available (ATCC) E. coli/S. gordonii shuttle plasmid VA838, as described in Warren et al., Protein Expr Purif 40:319-326 (2005), to produce enhanced secretion of heterologous proteins. This S. gordonii system is used to study the ability of in situ secreted VHH to protect against cell-associated transmission. In this system, cell-associated transmission can be isolated and studied independent of cell-free transmission in the presence of progesterone pre-treatment. Additionally, this strain is used to study the ability of in situ-produced anti-HSV-2 Gp D VHH to protect in the mouse herpes challenge model, which also requires progesterone pre-treatment.

6. Camelid-Derived VHH Fragments that Bind to CD18

a. Alpaca Immunization and VHH Production

Alpacas were immunized 4 times with 0.4 mg of GST-CD18 (see CD18 sequence supra attached to a GST tag) in two week intervals. Blood samples were taken before the first immunization and two weeks after each successive immunization. Alpaca lymph tissue was isolated and RNA was extracted. RNA was reverse transcribed into cDNA and vhh was amplified using primers against the leader region and hinge region surrounding vhh. PCR products were further amplified with primers containing restriction sites for insertion into T7 phage vectors. The resulting vhh was ligated into T7Select10-3b arms provided by Novagen and packaged into T7 phage. The size of the resulting library was 2.9×10⁷ pfu. This library was amplified for biopanning.

b. Biopanning and VHH Selection

His-CD18 was immobilized in a 96 well Maxisorp Nunc immunoplate and the phage library was added to each well. Wells were washed with 1×TBS with 0.1% Tween-20. Bound phage were eluted with 1% SDS and added to E. coli for further amplification. Amplified phage were used for another round of biopanning and this process was repeated for further enrichment of phage that bound to CD18. A summary of phage enrichment is shown in Table 3.

TABLE 3
Enrichment of phage binding to CD18 after successive rounds of biopanning

| Round of Biopanning | Phage Output (pfu) |
|---|---|
| 1 | 2.6 × 10⁴ |
| 2 | 5.5 × 10⁴ |
| 3 | 1.76 × 10⁵ |
| 4 | 2.56 × 10⁶ |
| 5 | 5.0 × 10⁷ |

After one round of panning, 2.6×10⁴ pfu bound to His-CD18. Phage were further enriched to 5.5×10⁴ pfu, 1.76×10⁵ pfu, 2.56×10⁶ pfu, and 5.0×10⁷ pfu after each successive round of biopanning.

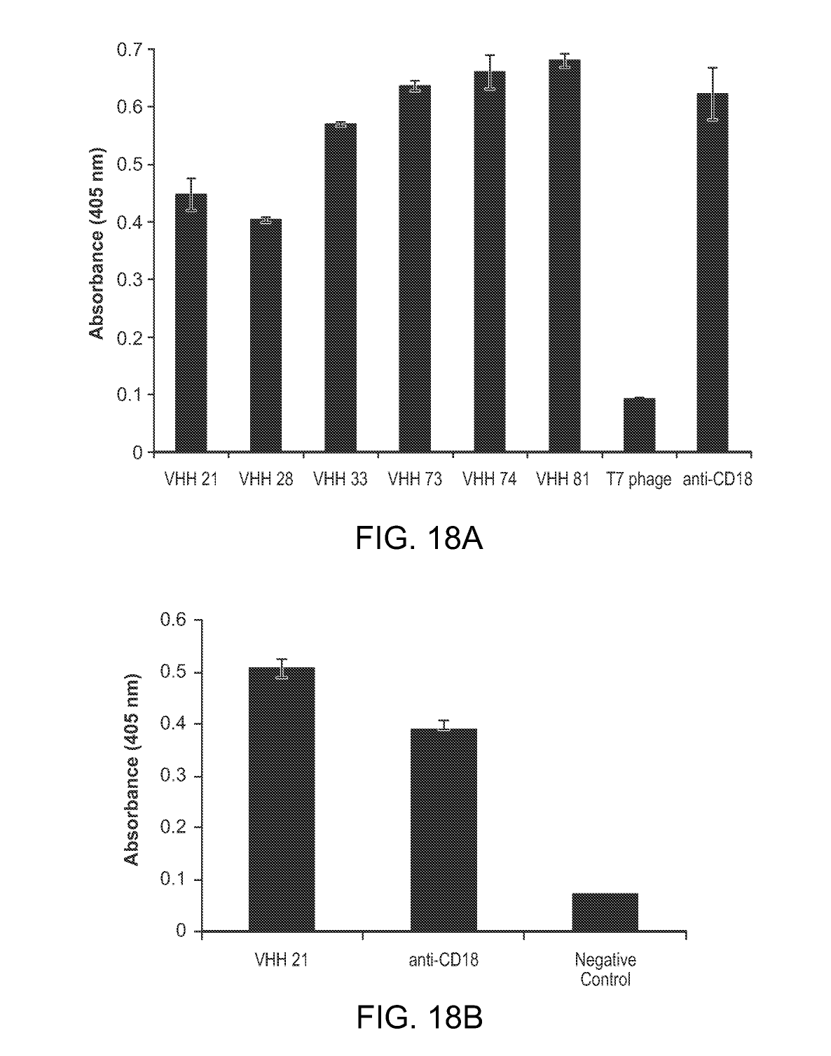

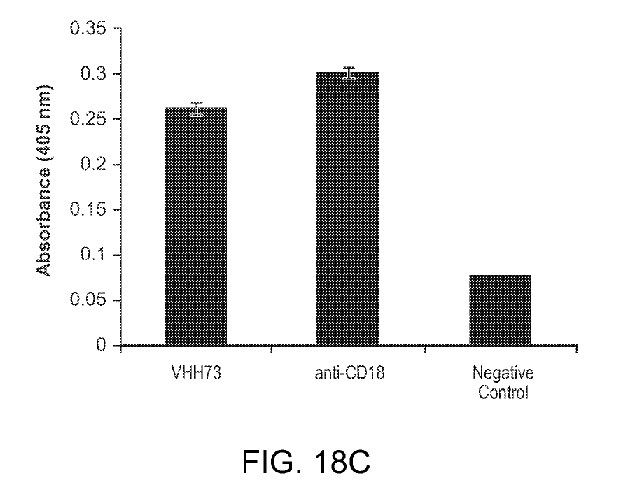

After the last round of panning, the eluted phage was added to E. coli and plated onto LB agar plates for plaque purification. Each population of phage was individually screened against His-CD18 for further selection using a phage ELISA. Individual amplified phage were added to His-CD18 immobilized onto Maxisorp Nunc immunoplate. Phage were detected by anti-T7 antibody and HRP-goat anti-mouse antibody. Out of 120 phage clones screened, 6 were positive (FIG. 18A).

c. VHH Expression, Purification, and Validation

Phage DNA was extracted from positive clones and amplified using T7 primers, and then sequenced. Out of 6 phage clones, 2 were unique (FIGS. 18B and 18C). These DNA sequences were cloned into pET47b containing a his tag. VHH was expressed and column purified using Ni-NTA agarose. VHH were validated using an ELISA against GSTCD18. Purified VHH were added to GST-CD18 immobilized onto Maxisorp Nunc immunoplate. Binding was detected using an anti-His antibody and HRP-goat anti-rabbit antibody.

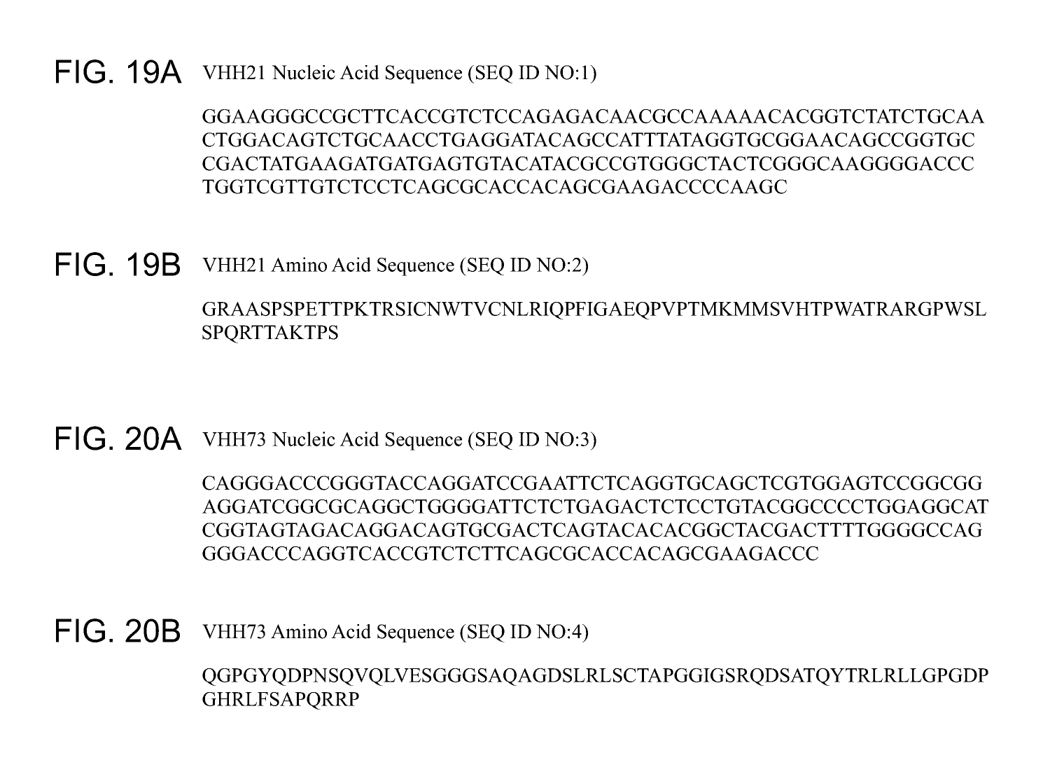

The sequences of the two clones, VHH21 and VHH73, are shown in FIGS. 19 and 20, respectively.

### Example 12

### VHH Targeting HSV Glycoprotein D

While glycoprotein-based vaccines against HSV-2 infection have shown only moderate efficacy in humans (Corey et al., Jama 282:331-340 (1999)), evidence from guinea pig models of the disease indicate that passive immunization can significantly ameliorate the course of the disease. Recent evidence that substantial Ab-mediated protection against genital HSV-2 disease could be achieved by either Fc-gammaR-dependent or -independent mechanisms provides a basis for pursuing technologies for in situ secretion of VHH (Chu et al., J Reprod Immunol 78:58-67 (2008)).

For these studies HSV-2 is obtained from ATCC, grown on Vero cells according to the recommendations of the ATCC, and viral RNA is isolated from the cells. The RNA is reverse transcribed, and cDNA is amplified using glycoprotein D specific primers 5′-GGAGGATCCAAATACTCCTTAGCA, which includes a Bam H1 restriction site, and 3′-ATTGAAGCTTGTTACGGGTTGCTGGGGGC, which includes a HindIII restriction site, for integration into the pQE30 plasmid, as modified from the method of described in van Kooij et al., Protein Expr Purif 25:400-408 (2002). The protein is purified using a nickel column and used for immunization of a camelid, as was done for CD18 and ICAM-1 above, for generation of VHH specific for glycoprotein D.

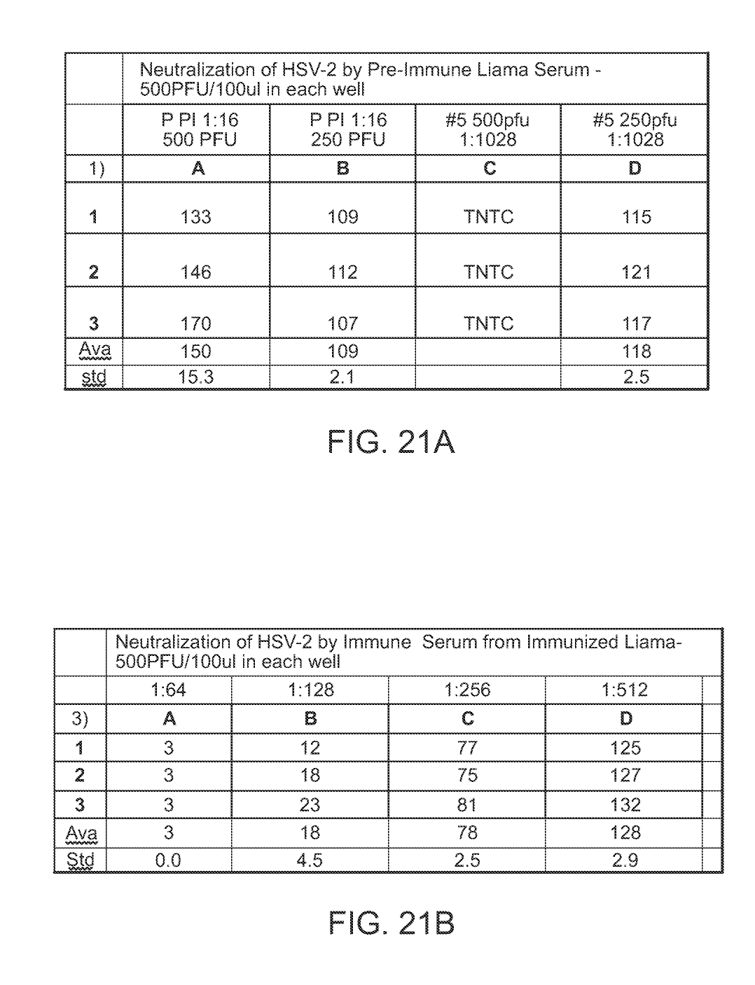

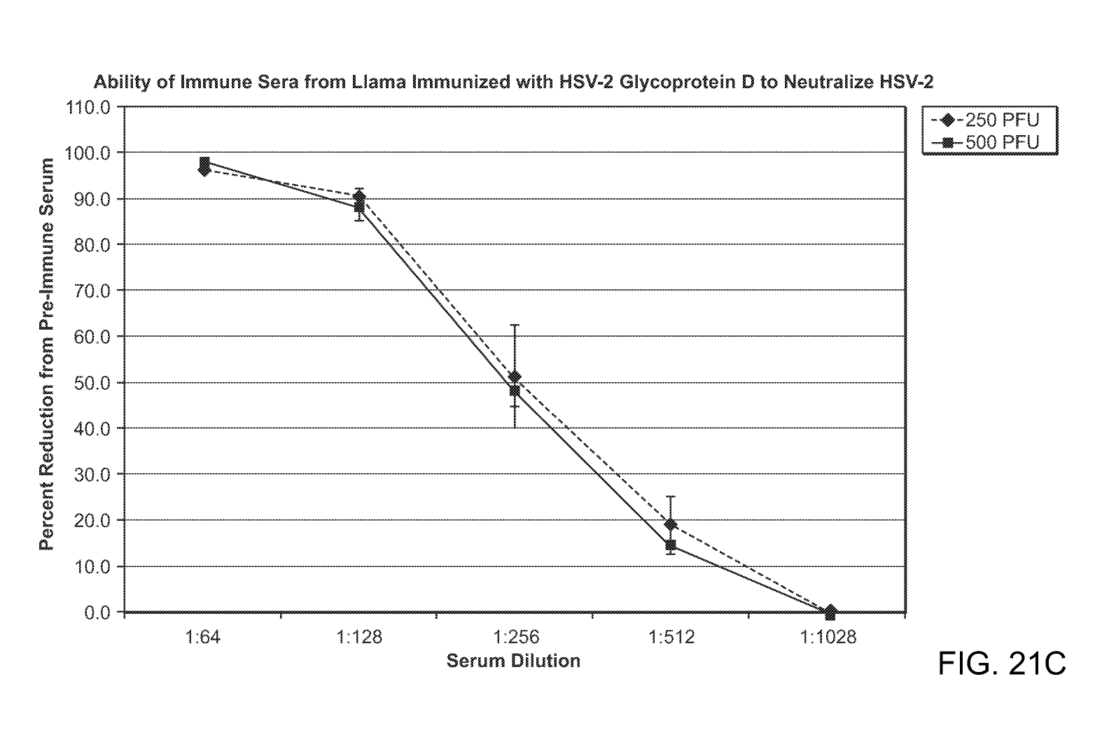

As shown in FIG. 21, llamas immunized with HSV-2 glycoprotein D produce neutralizing antibody that can be used in phage panning experiments.

### Example 13

### Testing the Ability of the to Block Transmission or Neutralize Infection in Transwell or In Vitro Neutralization Assays

The above-described ME180 cervical epithelial transwell culture system is used to evaluate the VHH. The ability of purified and concentrated VHH obtained from bacterial culture is assessed for its ability to block transepithelial transmission at different molar concentrations of the VHH.

In addition, VHH targeting gpD are also evaluated for their neutralization activity. A standard neutralization assay is performed (Zeitlin et al., J Reprod Immunol 40:93-101 (1998)). Briefly, serial dilutions of VHH over a two order of magnitude range, as described above, is incubated with 1500 TCID50 HSV-2, strain G (Virotech, Rockville, Md.) for 60 minutes at 37° C. in a total volume of 100 μl. The antibody-virus mixture is then be placed on target cells (human newborn foreskin diploid fibroblast cells from Bartels, Issaquah, Wash.) and CPE is scored at 48 hours. An irrelevant monoclonal antibody is used as a control for these studies.

### Example 14

### Evaluate In Situ Production of VHH by Engineered Bacteria

Mice are inoculated with varying doses of transformed lactobacilli or streptococci (10 ul of 10⁷-10⁹ CFU/ml) and vaginal lavages are performed at 2, 24, 48, 96, and 144 hours after inoculation to determine persistence of the bacteria in the vaginal cavity. Similarly concentrations of VIM are determined in the lavage fluid using standard immunoassays (e.g., Western blots with anti-E-tag antibody to detect the E-tag incorporated into the VHH). Upon confirmation of colonization and VHH production, the mice are challenged according to the protocols described in Chancey et al., J Immunol 176:5627-5636 (2006); Khanna et al., J Clin Invest 109:205-211 (2002); and Denton et al., PLoS Med 5:e16 (2008).

For HSV-2 D glycoprotein, the mouse models described in Whaley et al., J. Infect. Dis. 169:647-649 (1994); Zeitlin et al., J Reprod Immunol 40:93-101 (1998); Zeitlin et al., Virology 225:213-215 (1996); Chu et al., J Reprod Immunol 78:58-67 (2008); and Zeitlin et al., Clin. Immun. Immunopath. 76:S126 (1995), are used to evaluate the ability of varying doses of VHH, to protect against challenge by HSV-2. These mouse model systems involve progesterone pre-treatment of mice to enhance establishment of infection.

In addition, the concentration of VHH that can be obtained in situ in the in vivo setting is determined as follows. Although it enhances HSV-2 infection, administration of progesterone inhibits colonization of the mouse vagina by lactobacilli (unpublished observations), and estradiol treatment enhances colonization (de Ruiz et al., Biol Pharm Bull 24:127-134 (2001)). Because the lactobacillus strain used in these studies are not selected for growth in mice, the mice are pre-treated with estradiol. The following day, the mice are inoculated with 10⁷ colony-forming units of the transformed lactobacilli. Lactobacilli (administered in phosphate buffered saline) for inoculation are obtained from log phase growth in MRS broth with CFU having been previously correlated with OD in broth culture. At 4, 8, 16, 24 and 48 hours after inoculation, groups of 3 mice are anesthetized and vaginal lavage is performed. Lavage fluid is plated at different dilutions on MRS agar and placed into an anaerobic chamber at 37° C., and a filtrate of the fluid is used to determine the concentration of VHH recovered, as measured on Western blot. Thus, data is acquired at different intervals that give some idea of the correlation between bacterial colony counts and production of VHH.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

### Incorporation by Reference

All patents and publications mentioned in this specification are herein incorporated by reference to the same extent as if each independent patent and publication was specifically and individually indicated to be incorporated by reference.

1. A camelid-derived antibody that specifically binds to ICAM-1 or a fragment thereof.

2. The antibody of claim 1, wherein the antibody is isolated.

3. The antibody of claim 1, wherein the antibody is an antibody fragment.

4. The antibody of claim 1, wherein the antibody is humanized.

5. The antibody of claim 1, wherein the antibody inhibits viral migration in a transwell assay.

6. A cell producing the antibody of claim 1.

7-8. (canceled)

9. A pharmaceutical composition comprising the antibody of claim 1.

10-19. (canceled)

20. A camelid-derived antibody that specifically binds to CD18 or a fragment thereof.

21-24. (canceled)

25. A cell producing the antibody of claim 20.

26-27. (canceled)

28. A pharmaceutical composition comprising the antibody of claim 20.

29-38. (canceled)

39. A method of inhibiting the establishment or persistence of viral infection in a subject having or at risk of developing a viral infection, the method comprising contacting an epithelial cell with a camelid-derived antibody that specifically binds to ICAM-1, thereby inhibiting transepithelial transmission of the virus, or
A method of inhibiting viral transmission in a subject having or at risk of developing a viral infection, the method comprising contacting an epithelial cell with a camelid-derived antibody that specifically binds to ICAM-1, thereby inhibiting transepithelial transmission of the virus.

40. (canceled)

41. The method of claim 39, wherein the virus is HIV.

42-43. (canceled)

44. The method of claim 39, wherein the method further comprises delivering the antibody to the subject using a bacterial delivery system.

45-46. (canceled)

47. The method of claim 39, wherein the method further comprises administering a camelid-derived antibody that specifically binds to CD18.

48-51. (canceled)

52. A method of inhibiting the establishment or persistence of viral infection in a subject having or at risk of developing a viral infection, the method comprising contacting an epithelial cell with a camelid-derived antibody that specifically binds to CD18, thereby inhibiting transepithelial transmission of the virus, or
A method of inhibiting viral transmission in a subject having or at risk of developing a viral infection, the method comprising contacting an epithelial cell with a camelid-derived antibody that specifically binds to CD18, thereby inhibiting transepithelial transmission of the virus.

54. The method of claim 52, wherein the virus is HIV.

55-64. (canceled)

65. A bacterial cell that produces a camelid-derived antibody that specifically binds to ICAM-1 or a fragment thereof in situ, or
A bacterial cell that produces a camelid-derived antibody that specifically binds to CD18 or a fragment thereof in situ.

66. (canceled)

67. A pharmaceutical composition comprising the bacterial cell of claim 65.

68. A method of inhibiting the establishment or persistence of HIV-1 infection in a subject having or at risk of developing a viral infection, the method comprising contacting an epithelial cell with a camelid-derived antibody that specifically binds to CD18 and/or a camelid-derived antibody that specifically binds to ICAM-1, thereby inhibiting transepithelial transmission of the virus, or
A method of inhibiting HIV-1 transmission in a subject having or at risk of developing a viral infection, the method comprising contacting an epithelial cell with a camelid-derived antibody that specifically binds to CD18 and/or a camelid-derived antibody that specifically binds to ICAM-1, thereby inhibiting transepithelial transmission of the virus.

69-71. (canceled)

72. A camelid-derived antibody that specifically binds to HSV-2 glycoprotein D or a fragment thereof.

73-75. (canceled)

76. A bacterial cell that produces the antibody of claim 72.

77. A pharmaceutical composition comprising the antibody of claim 72.

78-81. (canceled)

82. A method of inhibiting the establishment or persistence of HSV-2 infection in a subject having or at risk of developing a viral infection, the method comprising contacting an epithelial cell with a camelid-derived antibody that specifically binds to HSV-2 glycoprotein D, thereby inhibiting transepithelial transmission of the virus.

83-86. (canceled)

87. A method of identifying an antibody or antibody fragment that specifically binds to ICAM-1, the method comprising panning a phage-display library, wherein the phage display library displays at least one peptide comprising the framework regions of a camelid-derived VHH or an amino acid sequence having at least 60%, 70%, 80%, or 90% sequence identity thereto, or
A method of identifying an antibody or antibody fragment that specifically binds to CD18, the method comprising panning a phage-display library, wherein the phage display library displays at least one peptide comprising the framework regions of a camelid-derived VHH or an amino acid sequence having at least 60%, 70%, 80%, or 90% sequence identity thereto, or
A method of identifying an antibody or antibody fragment that specifically binds to HSV-2 glycoprotein D, the method comprising panning a phage-display library, wherein the phage display library displays at least one peptide comprising the framework regions of a camelid-derived VHH or an amino acid sequence having at least 60%, 70%, 80%, or 90% sequence identity thereto.

88-91. (canceled)

92. An antibody or antibody fragment that specifically binds to CD18 obtained from the method of claim 87.

93-100. (canceled)


SEQUENCE LISTING (23 sequences)

SEQ ID NO: 1 - DNA, 210 bp, Artificial Sequence
  Description of Artificial Sequence Synthetic polynucleotide
ggaagggccgcttcaccgtctccagagacaacgccaaaaacacggtctatctgcaactggacagtctgcaacctgaggatacagccatttataggtgcggaacagccggtgccgactatgaagatgatgagtgtacatacgccgtgggctactcgggcaaggggaccctggtcgttgtctcctcagcgcaccacagcgaagaccccaagc

SEQ ID NO: 2 - protein, 70 aa, Artificial Sequence
  Description of Artificial Sequence Synthetic polypeptide
GRAASPSPETTPKTRSICNWTVCNLRIQPFIGAEQPVPTMKMMSVHTPWATRARGPWSLSPQRTTAKTPS

SEQ ID NO: 3 - DNA, 213 bp, Artificial Sequence
  Description of Artificial Sequence Synthetic polynucleotide
cagggacccgggtaccaggatccgaattctcaggtgcagctcgtggagtccggcggaggatcggcgcaggctggggattctctgagactctcctgtacggcccctggaggcatcggtagtagacaggacagtgcgactcagtacacacggctacgacttttggggccaggggacccaggtcaccgtctcttcagcgcaccacagcgaagaccc

SEQ ID NO: 4 - protein, 71 aa, Artificial Sequence
  Description of Artificial Sequence Synthetic polypeptide
QGPGYQDPNSQVQLVESGGGSAQAGDSLRLSCTAPGGIGSRQDSATQYTRLRLLGPGDPGHRLFSAPQRRP

SEQ ID NO: 5 - DNA, 1846 bp, Homo sapiens
ctcagcctcgctatggctcccagcagcccccggcccgcgctgcccgcactcctggtcctgctcggggctctgttcccaggacctggcaatgcccagacatctgtgtccccctcaaaagtcatcctgccccggggaggctccgtgctggtgacatgcagcacctcctgtgaccagcccaagttgttgggcatagagaccccgttgcctaaaaaggagttgctcctgcctgggaacaaccggaaggtgtatgaactgagcaatgtgcaagaagatagccaaccaatgtgctattcaaactgccctgatgggcagtcaacagctaaaaccttcctcaccgtgtactggactccagaacgggtggaactggcacccctcccctcttggcagccagtgggcaagaaccttaccctacgctgccaggtggagggtggggcaccccgggccaacctcaccgtggtgctgctccgtggggagaaggagctgaaacgggagccagctgtgggggagcccgctgaggtcacgaccacggtgctggtgaggagagatcaccatggagccaatttctcgtgccgcactgaactggacctgcggccccaagggctggagctgtttgagaacacctcggccccctaccagctccagacctttgtcctgccagcgactcccccacaacttgtcagcccccgggtcctagaggtggacacgcaggggaccgtggtctgttccctggacgggctgttcccagtctcggaggcccaggtccacctggcactgggggaccagaggttgaaccccacagtcacctatggcaacgactccttctcggccaaggcctcagtcagtgtgaccgcagaggacgagggcacccagcggctgacgtgtgcagtaatactggggaaccagagccaggagacactgcagacagtgaccatctacagctttccggcgcccaacgtgattctgacgaagccagaggtctcagaagggaccgaggtgacagtgaagtgtgaggcccaccctagagccaaggtgacgctgaatggggttccagcccagccactgggcccgagggcccagctcctgctgaaggccaccccagaggacaacgggcgcagcttctcctgctctgcaaccctggaggtggccggccagcttatacacaagaaccagacccgggagcttcgtgtcctgtatggcccccgactggacgagagggattgtccgggaaactggacgtggccagaaaattcccagcagactccaatgtgccaggcttgggggaacccattgcccgagctcaagtgtctaaaggatggcactttcccactgcccatcggggaatcagtgactgtcactcgagatcttgagggcacctacctctgtcgggccaggagcactcaaggggaggtcacccgcgaggtgaccgtgaatgtgctctccccccggtatgagattgtcatcatcactgtggtagcagccgcagtcataatgggcactgcaggcctcagcacgtacctctataaccgccagcggaagatcaagaaatacagactacaacaggcccaaaaagggacccccatgaaaccgaacacacaagccacgcctccctgaacctatcccgggacagggcctcttcctcggccttcccatattggtggcagtggtgccacactgaacagagtggaagacatatgccatgcagctacacctaccggccctgggacgccggaggacagggcattgtcctcagtcagatacaacagcatttggggccatggtacctgcacacctaaaacactaggccacgcatctgatctgtagtcacatgactaagccaagaggaagg

SEQ ID NO: 6 - protein, 532 aa, Homo sapiens
MAPSSPRPALPALLVLLGALFPGPGNAQTSVSPSKVILPRGGSVLVTCSTSCDQPKLLGIETPLPKKELLLPGNNRKVYELSNVQEDSQPMCYSNCPDGQSTAKTFLTVYWTPERVELAPLPSWQPVGKNLTLRCQVEGGAPRANLTVVLLRGEKELKREPAVGEPAEVTTTVLVRRDHHGANFSCRTELDLRPQGLELFENTSAPYQLQTFVLPATPPQLVSPRVLEVDTQGTVVCSLDGLFPVSEAQVHLALGDQRLNPTVTYGNDSFSAKASVSVTAEDEGTQRLTCAVILGNQSQETLQTVTIYSFPAPNVILTKPEVSEGTEVTVKCEAHPRAKVTLNGVPAQPLGPRAQLLLKATPEDNGRSFSCSATLEVAGQLIHKNQTRELRVLYGPRLDERDCPGNWTWPENSQQTPMCQAWGNPLPELKCLKDGTFPLPIGESVTVTRDLEGTYLCRARSTQGEVTREVTVNVLSPRYEIVIITVVAAAVIMGTAGLSTYLYNRQRKIKKYRLQQAQKGTPMKPNTQATPP

SEQ ID NO: 7 - DNA, 687 bp, Artificial Sequence
  Description of Artificial Sequence Synthetic polynucleotide
ctgcgaccaggccaggcagcagcgttcaacgtgaccttccggcgggccaagggctaccccatcgacctgtactatctgatggacctctcctactccatgcttgatgacctcaggaatgtcaagaagctaggtggcgacctgctccgggccctcaacgagatcaccgagtccggccgcattggcttcgggtccttcgtggacaagaccgtgctgccgttcgtgaacacgcaccctgataagctgcgaaacccatgccccaacaaggagaaagagtgccagcccccgtttgccttcaggcacgtgctgaagctgaccaacaactccaaccagtttcagaccgaggtcgggaagcagctgatttccggaaacctggatgcacccgagggtgggctggacgccatgatgcaggtcgccgcctgcccggaggaaatcggctggcgcaacgtcacgcggctgctggtgtttgccactgatgacggcttccatttcgcgggcgacggaaagctgggcgccatcctgacccccaacgacggccgctgtcacctggaggacaacttgtacaagaggagcaacgaattcgactacccatcggtgggccagctggcgcacaagctggctgaaaacaacatccagcccatcttcgcggtgaccagtaggatggtgaagacctacgagaaactcaccgag

SEQ ID NO: 8 - protein, 229 aa, Artificial Sequence
  Description of Artificial Sequence Synthetic polypeptide
LRPGQAAAFNVTFRRAKGYPIDLYYLMDLSYSMLDDLRNVKKLGGDLLRALNEITESGRIGFGSFVDKTVLPFVNTHPDKLRNPCPNKEKECQPPFAFRHVLKLTNNSNQFQTEVGKQLISGNLDAPEGGLDAMMQVAACPEEIGWRNVTRLLVFATDDGFHFAGDGKLGAILTPNDGRCHLEDNLYKRSNEFDYPSVGQLAHKLAENNIQPIFAVTSRMVKTYEKLTE

SEQ ID NO: 9 - DNA, 942 bp, Artificial Sequence
  Description of Artificial Sequence Synthetic polynucleotide
aaatacgccttagcagacccctcgcttaagatggccgatcccaatcgatttcgcgggaagaaccttccggttttggaccagctgaccgacccccccggggtgaagcgtgtttaccacattcagccgagcctggaggacccgttccagccccccagcatcccgatcactgtgtactacgcagtgctggaacgtgcctgccgcagcgtgctcctacatgccccatcggaggccccccagatcgtgcgcggggcttcggacgaggcccgaaagcacacgtacaacctgaccatcgcctggtatcgcatgggagacaattgcgctatccccatcacggttatggaatacaccgagtgcccctacaacaagtcgttgggggtctgccccatccgaacgcagccccgctggagctactatgacagctttagcgccgtcagcgaggataacctgggattcctgatgcacgcccccgccttcgagaccgcgggtacgtacctgcggctagtgaagataaacgactggacggagatcacacaatttatcctggagcaccgggcccgcgcctcctgcaagtacgctctccccctgcgcatccccccggcagcgtgcctcacctcgaaggcctaccaacagggcgtgacggtcgacagcatcgggatgctaccccgctttatccccgaaaaccagcgcaccgtcgccctatacagcttaaaaatcgccgggtggcacggccccaagcccccgtacaccagcaccctgctgccgccggagctgtccgacaccaccaacgccacgcaacccgaactcgttccggaagaccccgaggactcggccctcttagaggatcccgccgggacggtgtcttcgcagatccccccaaactggcacatcccgtcgatccaggacgtcgcgccgcaccacgcccccgccgcccccagcaacccg

SEQ ID NO: 10 - protein, 314 aa, Artificial Sequence
  Description of Artificial Sequence Synthetic polypeptide
KYALADPSLKMADPNRFRGKNLPVLDQLTDPPGVKRVYHIQPSLEDPFQPPSIPITVYYAVLERACRSVLLHAPSEAPQIVRGASDEARKHTYNLTIAWYRMGDNCAIPITVMEYTECPYNKSLGVCPIRTQPRWSYYDSFSAVSEDNLGFLMHAPAFETAGTYLRLVKINDWTEITQFILEHRARASCKYALPLRIPPAACLTSKAYQQGVTVDSIGMLPRFIPENQRTVALYSLKIAGWHGPKPPYTSTLLPPELSDTTNATQPELVPEDPEDSALLEDPAGTVSSQIPPNWHIPSIQDVAPHHAPAAPSNP

SEQ ID NO: 11 - DNA, 23 bp, Artificial Sequence
  Description of Artificial Sequence Synthetic primer
gaggtbcarctgcaggasagygg

SEQ ID NO: 12 - DNA, 23 bp, Artificial Sequence
  Description of Artificial Sequence Synthetic primer
gaggtbcarctgcaggastcygg

SEQ ID NO: 13 - protein, 6 aa, Artificial Sequence
  Description of Artificial Sequence Synthetic 6xHis tag
HHHHHH

SEQ ID NO: 14 - DNA, 30 bp, Artificial Sequence
  Description of Artificial Sequence Synthetic primer
gatcagatctctgcgaccaggccaggcagc

SEQ ID NO: 15 - DNA, 29 bp, Artificial Sequence
  Description of Artificial Sequence Synthetic primer
ctagtcgacctcggtgagtttctcgtagg

SEQ ID NO: 16 - DNA, 29 bp, Artificial Sequence
  Description of Artificial Sequence Synthetic primer
ccaggatccctgcgaccaggccaggcagc

SEQ ID NO: 17 - DNA, 34 bp, Artificial Sequence
  Description of Artificial Sequence Synthetic primer
cggaagcttttactcggtgagtttctcgtaggtc

SEQ ID NO: 18 - DNA, 18 bp, Artificial Sequence
  Description of Artificial Sequence Synthetic oligonucleotide
tttttttttttttttttt

SEQ ID NO: 19 - DNA, 30 bp, Artificial Sequence
  Description of Artificial Sequence Synthetic primer
gatcactagtggggtcttcgctgtggtgcg

SEQ ID NO: 20 - DNA, 31 bp, Artificial Sequence
  Description of Artificial Sequence Synthetic primer
gatcactagtttgtggttttggtgtcttggg

SEQ ID NO: 21 - DNA, 36 bp, Artificial Sequence
  Description of Artificial Sequence Synthetic primer
gatcgccggccagktgcagctcgtggagtcnggngg

SEQ ID NO: 22 - DNA, 24 bp, Artificial Sequence
  Description of Artificial Sequence Synthetic primer
ggaggatccaaatactccttagca

SEQ ID NO: 23 - DNA, 29 bp, Artificial Sequence
  Description of Artificial Sequence Synthetic primer
attgaagcttgttacgggttgctgggggc